(19) United States
(12) Patent Application Publication
(10) Pub. No.: US 2002/0151534 A1
(43) Pub. Date: Oct. 17, 2002

(54) Antithrombotic compounds

(76) Inventors: Uwe Joerg Ries, Biberach (DE); Henning Priepke, Warthausen (DE); Herbert Nar, Ochsenhausen (DE); Jean-Marie Stassen, Leuven (BE); Wolfgang Wienen, Biberach/Rissegg (DE)

Correspondence Address: BOEHRINGER INGELHEIM CORPORATION, 900 RIDGEBURY ROAD, P. O. BOX 368, RIDGEFIELD, CT, 06877, US

(21) Appl. No.: 10/050,376

(22) Filed: Jan. 16, 2002

Related U.S. Application Data
(60) Provisional application No. 60/268,569, filed on Feb. 15, 2001.

(30) Foreign Application Priority Data
Feb. 2, 2001 (DE) DE 101 04 597.2
Jul. 26, 2001 (DE) DE 101 36 435.0

(51) Int. Cl. A61K 31/397; A61K 31/55; A61K 31/54; A61K 31/535; A61K 31/445; C07D 211/32

(52) U.S. Cl. 514/210.01; 514/217.12; 514/227.5; 514/231.2; 514/317; 514/408; 514/617; 544/59; 544/170; 546/233; 548/578; 548/950

(57) ABSTRACT

Antithrombotic compounds of general formula

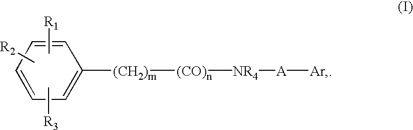

Exemplary are:

(1) 2-(5-carbamimidoyl-2-hydroxy-phenyl)-N-[3-methyl-4-(pyrrolidin-1-yl-carbonyl)-phenyl]-ethylamine,

(2) N-(5-carbamimidoyl-2-hydroxy-benzyl)-3-methyl-4-(pyrrolidin-1-yl-carbonyl)-benzylamine, and

(3) N-(5-carbamimidoyl-2-hydroxy-benzyl)-2,5-dimethyl-4-(pyrrolidin-1-yl-carbonyl)-benzylamine.

### RELATED APPLICATIONS

Benefit of U.S. Provisional Application Serial No. 60/268,569, filed on Feb. 15, 2001is hereby claimed.

### DESCRIPTION OF THE INVENTION

The present invention relates to the compounds of general formula

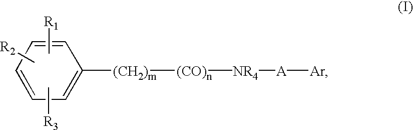

the tautomers, the stereoisomers, the mixtures, the prodrugs, the derivatives thereof which contain a group that is negatively charged under physiological conditions instead of a carboxy group, and the salts thereof, particularly the physiologically acceptable salts thereof with inorganic or organic acids or bases which have valuable properties.

The compounds of the above general formula I wherein Ar denotes a phenyl or naphthyl group substituted by the groups R₅, R₆ and R₇, and R₅ denotes a cyano group, are valuable intermediate products for preparing the corresponding compounds of general formula I wherein R₅ denotes an amidino group optionally substituted by one or two C₁₋₃-alkyl groups. The compounds of the above general formula I with the exception of those compounds wherein Ar denotes a phenyl or naphthyl group substituted by the groups R₅, R₆ and R₇, and R₅ denotes a cyano group, as well as the tautomers, the stereoisomers, the mixtures, the prodrugs, the derivatives thereof which contain a group that is negatively charged under physiological conditions instead of a carboxy group, and the salts thereof, particularly the physiologically acceptable salts thereof with inorganic or organic acids, and the stereoisomers thereof, have valuable pharmacological properties, particularly an antithrombotic activity and an inhibiting effect on factor Xa.

The present application thus relates to the new compounds of the above general formula I and the preparation thereof, the pharmaceutical compositions containing the pharmacologically effective compounds, their preparation and use.

In the above general formula

(i) m denotes the number 0,

n denotes the number 1 and

A denotes a straight-chain C₁₋₃-alkylene group wherein

one or two hydrogen atoms independently of one another may be replaced in each case by a C₁₋₃-alkyl group or

a hydrogen atom may be replaced by the group '(CH₂)ₚ—R_{f}, while

p denotes one of the numbers 0, 1, 2 or 3 and

R_{f} denotes a hydroxycarbonyl, C₁₋₃-alkoxycarbonyl, aminocarbonyl, C₁₋₃-alkylaminocarbonyl, di-(C₁₋₃-alkyl)-aminocarbonyl, C₃₋₇-cycloalkylamino-carbonyl, N-(C₁₋₃-alkoxy-carbonylmethyl)-N-(C₁₋₃-alkyl)-aminocarbonyl, N-(carboxymethyl)-N-(C₁₋₃-alkyl)-aminocarbonyl or a 4- to 7-membered cycloalkyleneimino-carbonyl group, or

(ii) m denotes the number 1,

n denotes the number 1 and

A denotes a bond or

(iii) m denotes the number 0 or 1,

n denotes the number 0 and

A denotes a straight-chain C₁₋₃-alkylene group wherein one or two hydrogen atoms independently of one another may be replaced in each case by a C₁₋₃-alkyl group, or

(iv) m denotes the number 2,

n denotes the number 0 and

A denotes a bond,

R₁ denotes an amino, C₁₋₅-alkylamino, C₃₋₇-cycloalkylamino or phenyl-C₁₋₃-alkylamino group each of which may be substituted at the amino nitrogen atom by a phenylcarbonyl or phenylsulphonyl group or by a C₁₋₃-alkyl or C₁₋₃-alkyl-carbonyl group optionally substituted in the alkyl moiety by a carboxy group, a group which may be converted in vivo into a carboxy group, an amino, C₁₋₃-alkylamino or di-(C₁₋₃-alkyl)-amino group,

a di-(C₁₋₅-alkyl)amino or N-(C₃₋₇-cycloalkyl)-C₁₋₅-alkylamino group, while the C₁₋₅-alkyl moiety with the exception of the 1 position may be substituted in each case by a hydroxy, C₁₋₃-alkoxy, amino, C₁₋₃-alkyl-amino or di-(C₁₋₃-alkyl)-amino group,

a 4- to 7-membered cycloalkyleneiminocarbonyl or cycloalkyleneiminosulphonyl group optionally substituted by a C₁₋₃-alkyl, amino-C₁₋₃-alkyl, C₁₋₃-alkylamino-C₁₋₃-alkyl, di-(C₁₋₃-alkyl)-amino-C₁₋₃-alkyl, aminocarbonyl, C₁₋₃-alkylamino-carbonyl or di-(C₁₋₃-alkyl)-aminocarbonyl group,

a 2,5-dihydropyrrol-1-yl-carbonyl group,

an aminosulphonyl group optionally substituted by one or two C₁₋₃-alkyl groups,

a C₃₋₇-cycloalkyl-carbonyl group, whilst

the methylene group in the 3 or 4 position in a C₅₋₇-cycloalkyl-carbonyl group may be replaced by an —NH group wherein

the hydrogen atom of the —NH group may be replaced by a C₁₋₃-alkyl, C₁₋₃-alkyl-carbonyl, phenylcarbonyl or phenylsulphonyl group,

a phenylcarbonyl or heteroarylcarbonyl group,

or a C₁₋₃-alkyl group optionally monosubstituted by an amino, C₁₋₃-alkylamino, di-(C₁₋₃-alkyl)-amino, hydroxy, phenyl or a 4- to 7-membered cycloalkyleneimino group or terminally disubstituted by a phenyl group and a hydroxy group, while

the phenyl substituents may be substituted by an amidino group optionally substituted by one or two C₁₋₃-alkyl groups, by a fluorine, chlorine or bromine atom, by a trifluoromethyl, C₁₋₃-alkyl or C₁₋₃-alkoxy group,

R₂ denotes a hydrogen, fluorine, chlorine or bromine atom, a C₁₋₃-alkyl group wherein the hydrogen atoms may be wholly or partly replaced by fluorine atoms, a C₂₋₃-alkenyl, C₂₋₃-alkynyl, hydroxy, C₁₋₃-alkoxy or trifluoromethoxy group,

R₃ denotes a hydrogen atom or a C₁₋₃-alkyl group,

R₄ denotes a hydrogen atom or a C₁₋₃-alkyl group optionally substituted by a carboxy group or a group which may be converted in vivo into a carboxy group and

Ar denotes a phenyl or naphthyl group substituted by the groups R₅, R₆ and R₇, while

R₅ denotes a cyano group, an amidino group optionally substituted by one or two C₁₋₃-alkyl groups, an amino-C₁₋₃-alkyl, C₁₋₃-alkylamino-C₁₋₃-alkyl or di-(C₁₋₃-alkyl)amino-C₁₋₃-alkyl group,

R₆ denotes a hydrogen, fluorine, chlorine or bromine atom, a trifluoromethyl, C₁₋₃-alkyl, hydroxy, hydroxy-C₁₋₃-alkyl, C₁₋₃-alkoxy, C₁₋₃-alkoxy-C₁₋₃-alkyl, carboxy, carboxy-C₁₋₃-alkyl, carboxy-C₁₋₃-alkoxy, C₁₋₄-alkoxy-carbonyloxy, C₁₋₄-alkoxy-carbonyl-C₁₋₃-alkoxy, phenyl-C₁₋₃-alkoxy, amino, C₁₋₃-alkylamino or di-(C₁₋₃-alkyl)amino group and

R₇ denotes a hydrogen, fluorine, chlorine or bromine atom or a C₁₋₃-alkyl group,

or a thienyl, thiazolyl, pyridinyl, pyrimidinyl, pyrazinyl or pyridazinyl group optionally substituted in the carbon skeleton by a C₁₋₃-alkyl group,

while the term heteroaryl group mentioned above denotes a 5-membered heteroaryl group bound via a carbon or nitrogen atom which contains

an imino group optionally substituted by a C₁₋₄-alkyl or C₁₋₄-alkyl-carbonyl group, an oxygen or sulphur atom,

an imino group optionally substituted by a C₁₋₄-alkyl group or an oxygen or sulphur atom and additionally a nitrogen atom,

an imino group optionally substituted by a C₁₋₄-alkyl group and two nitrogen atoms or

an oxygen or sulphur atom and two nitrogen atoms,

or a 6-membered heteroaryl group which contains one or two nitrogen atoms,

while a phenyl ring may be fused to the abovementioned 5- or 6-membered heteroaryl groups via two adjacent carbon atoms and the bicyclic heteroaryl groups thus formed may be bound via the heteroaromatic or carbocyclic moiety, and the unsubstituted or monosubstituted phenyl groups mentioned in the definition of the abovementioned groups, or the unsubstituted or monosubstituted phenyl moieties contained in these groups, as well as the abovementioned heteroaryl groups may additionally be substituted at a carbon atom in each case by a fluorine, chlorine or bromine atom, by a trifluoromethyl, C₁₋₃-alkyl or C₁₋₃-alkoxy group, unless otherwise stated.

The carboxy groups mentioned in the definition of the abovementioned groups may be replaced by a group which may be converted in vivo into a carboxy group or by a group which is negatively charged under physiological conditions,

and moreover the amino and imino groups mentioned in the definition of the abovementioned groups may be substituted by a group which can be cleaved in vivo. Such groups are described for example in WO 98/46576 and by N. M. Nielsen et al. in Inter-national Journal of Pharmaceutics 39, 75-85 (1987).

By a group which can be converted in vivo into a carboxy group is meant, for example, a hydroxymethyl group, a carboxy group esterified with an alcohol wherein the alcohol moiety is preferably a C₁₋₆-alkanol, a phenyl-C₁₋₃-alkanol, a C₃₋₉-cycloalkanol, while a C₅₋₈-cycloalkanol may additionally be substituted by one or two C₁₋₃-alkyl groups, a C₅₋₈-cycloalkanol wherein a methylene group in the 3 or 4 position is replaced by an oxygen atom or by an imino group optionally substituted by a C₁₋₃-alkyl, phenyl-C₁₋₃-alkyl, phenyl-C₁₋₃-alkoxycarbonyl or C₂₋₆-alkanoyl group and the cycloalkanol moiety may additionally be substituted by one or two C₁₋₃-alkyl groups, a C₄₋₇-cycloalkenol, a C₃₋₅-alkenol, a phenyl-C₃₋₅-alkenol, a C₃₋₅-alkynol or phenyl-C₃₋₅-alkynol with the proviso that no bonds to the oxygen atom start from a carbon atom which carries a double or triple bond, a C₃₋₈-cycloalkyl-C₁₋₃-alkanol, a bicycloalkanol with a total of 8 to 10 carbon atoms which may additionally be substituted in the bicycloalkyl moiety by one or two C₁₋₃-alkyl groups, a 1,3-dihydro-3-oxo-1-isobenzofuranol or an alcohol of formula

Rₐ—CO—O—(R_{b}CR_{c})—OH,

wherein

Rₐ denotes a C₁₋₈-alkyl, C₅₋₇-cycloalkyl, phenyl or phenyl-C₁₋₃-alkyl group,

R_{b} denotes a hydrogen atom, a C₁₋₃-alkyl, C₅₋₇-cycloalkyl or phenyl group and

R_{c} denotes a hydrogen atom or a C₁₋₃-alkyl group,

by a group which is negatively charged under physiological conditions is meant, for example, a tetrazol-5-yl, phenylcarbonylaminocarbonyl, trifluoromethylcarbonylaminocarbonyl, C₁₋₆-alkylsulphonylamino, phenylsulphonylamino, benzylsulphonylamino, trifluoromethylsulphonylamino, C₁₋₆-alkylsulphonylaminocarbonyl, phenylsulphonylaminocarbonyl, benzylsulphonylaminocarbonyl or perfluoro-C₁₋₆-alkylsulphonylaminocarbonyl group

and by a group which can be cleaved in vivo from an imino or amino group is meant, for example, a hydroxy group, an acyl group such as a phenylcarbonyl group optionally mono- or disubstituted by fluorine, chlorine, bromine or iodine atoms, by C₁₋₃-alkyl or C₁₋₃-alkoxy groups, while the substituents may be identical or different, a pyridinoyl group or a C₁₋₁₆-alkanoyl group such as the formyl, acetyl, propionyl, butanoyl, pentanoyl or hexanoyl group, a 3,3,3-trichloropropionyl or allyloxycarbonyl group, a C₁₋₁₆-alkoxycarbonyl or C₁₋₁₆-alkylcarbonyloxy group, wherein hydrogen atoms may be wholly or partially replaced by fluorine or chlorine atoms such as the methoxycarbonyl, ethoxycarbonyl, propoxycarbonyl, isopropoxycarbonyl, butoxycarbonyl, tert.butoxycarbonyl, pentoxycarbonyl, hexyloxycarbonyl, octyloxycarbonyl, nonyloxycarbonyl, decyloxycarbonyl, undecyloxycarbonyl, dodecyloxycarbonyl, hexadecyloxycarbonyl, methylcarbonyloxy, ethylcarbonyloxy, 2,2,2-trichloroethylcarbonyloxy, propylcarbonyloxy, isopropylcarbonyloxy, butylcarbonyloxy, tert.butylcarbonyloxy, pentylcarbonyloxy, hexylcarbonyloxy, octylcarbonyloxy, nonylcarbonyloxy, decylcarbonyloxy, undecylcarbonyloxy, dodecylcarbonyloxy or hexadecylcarbonyloxy group, a phenyl-C₁₋₁₆-alkoxycarbonyl group such as the benzyloxycarbonyl, phenylethoxycarbonyl or phenylpropoxycarbonyl group, a 3-amino-propionyl group wherein the amino group may be mono- or disubstituted by C₁₋₆-alkyl or C₃₋₇-cycloalkyl groups and the substituents may be identical or different, a C₁₋₃-alkylsulphonyl-C₂₋₄-alkoxycarbonyl, C₁₋₃-alkoxy-C₂₋₄-alkoxy-C₂₋₄-alkoxycarbonyl, Rₐ—CO—O—(R_{b}CR_{c})—O—CO—, C₁₋₆-alkyl-CO—NH—(R_{d}CR_{c})—O—CO— or C₁₋₆-alkyl-CO—O—(R_{d}CRₑ)—(R_{d}CRₑ)—O—CO— group, wherein Rₐ to R_{c} are as hereinbefore defined,

R_{d} and Rₑ which may be identical or different, denote hydrogen atoms or C₁₋₃-alkyl groups.

Moreover, the saturated alkyl and alkoxy moieties containing more than 2 carbon atoms mentioned in the definitions above also include the branched isomers thereof such as the isopropyl, tert.butyl, isobutyl group, etc.

Preferred compounds of the above general formula I are those wherein

(i) m denotes the number 0,

n denotes the number 1 and

A denotes a straight-chain C₁₋₃-alkylene group wherein

one or two hydrogen atoms independently of one another may be replaced in each case by a C₁₋₃-alkyl group or

a hydrogen atom may be replaced by the group —(CH₂)ₚ—R_{f}, while

p denotes one of the numbers 0, 1, 2 or 3 and

R_{f} denotes a hydroxycarbonyl, C₁₋₃-alkoxycarbonyl, aminocarbonyl, C₁₋₃-alkylaminocarbonyl, di-(C₁₋₃-alkyl)-aminocarbonyl, C₃₋₇-cycloalkylamino-carbonyl, N-(C₁₋₃-alkoxy-carbonylmethyl)-N-(C₁₋₃-alkyl)-aminocarbonyl, N-(carboxymethyl)-N-(C₁₋₃-alkyl)-aminocarbonyl or a 4- to 7-membered cycloalkyleneimino-carbonyl group, or

(ii) m denotes the number 0 or 1,

n denotes the number 0 and

A denotes a straight-chain C₁₋₃-alkylene group wherein one or two hydrogen atoms independently of one another may be replaced in each case by a C₁₋₃-alkyl group,

R₁ denotes an amino, C₁₋₃-alkylamino or C₃₋₇-cycloalkylamino group each of which may be substituted at the amino nitrogen atom by a C₁₋₃-alkyl, C₁₋₃-alkylcarbonyl, carboxy-C₁₋₃-alkyl, carboxy-C₁₋₃-alkylcarbonyl, C₁₋₆-alkoxy-carbonyl-C₁₋₃-alkyl-carbonyl or amino-C₁₋₃-alkyl-carbonyl group,

a di-(C₁₋₃-alkyl)amino or N-(C₅₋₇-cycloalkyl)-C₁₋₃-alkylamino group,

a 4- to 7-membered cycloalkyleneiminocarbonyl group optionally substituted by a C₁₋₃-alkyl, amino-C₁₋₃-alkyl, C₁₋₃-alkylamino-C₁₋₃-alkyl, aminocarbonyl or C₁₋₃-alkylamino-carbonyl group, while

a hydrogen atom bound to a nitrogen atom may be replaced by an acetyl, phenylcarbonyl or tert.-butoxycarbonyl group,

or a 2,5-dihydropyrrol-1-yl-carbonyl group,

R₂ denotes a hydrogen, fluorine, chlorine or bromine atom, a C₁₋₃-alkyl, C₂₋₃-alkenyl, C₂₋₃-alkynyl, trifluoromethyl, C₁₋₃-alkoxy or trifluoromethoxy group,

R₃ denotes a hydrogen atom or a C₁₋₃-alkyl group,

R₄ denotes a hydrogen atom or a C₁₋₃-alkyl group and

Ar denotes a phenyl group substituted by the groups R₅, R₆ and R₇, while

R₅ denotes a cyano group, an amidino group optionally substituted by one or two C₁₋₃-alkyl groups, a hydroxy, C₁₋₆-alkoxy-carbonyl, 2,2,2-trichloroethoxycarbonyl or phenylcarbonyl group, or an amino-C₁₋₃-alkyl or C₁₋₃-alkylamino-C₁₋₃-alkyl group,

R₆ denotes a hydrogen, fluorine, chlorine or bromine atom, a trifluoromethyl, C₁₋₃-alkyl, hydroxy, hydroxy-C₁₋₃-alkyl, C₁₋₃-alkoxy, carboxy, C₁₋₃-alkoxy-carbonyloxy, carboxy-C₁₋₃-alkoxy or C₁₋₄-alkoxy-carbonyl-C₁₋₃-alkoxy group and

R₇ denotes a hydrogen atom or a C₁₋₃-alkyl group,

while the unsubstituted or monosubstituted phenyl groups mentioned in the definition of the abovementioned groups, or the unsubstituted or monosubstituted phenyl moieties contained in these groups, as well as the abovementioned heteroaryl groups may additionally be substituted at a carbon atom in each case by a fluorine, chlorine or bromine atom, by a trifluoromethyl, C₁₋₃-alkyl or C₁₋₃-alkoxy group, unless otherwise stated,

but particularly those compounds wherein

(i) m denotes the number 0,

n denotes the number 1 and

A denotes a methylene group wherein

one or two hydrogen atoms independently of one another may be replaced in each case by a C₁₋₃-alkyl group or

a hydrogen atom may be replaced by the group —(CH₂)ₚ—R_{f}, while

p denotes one of the numbers 0, 1, 2 or 3 and

R_{f} denotes a hydroxycarbonyl, C₁₋₃-alkoxycarbonyl, N-(C₁₋₃-alkyl)-amino-carbonyl, di-(C₁₋₃-alkyl)-aminocarbonyl, N-(C₁₋₃-alkoxy-carbonylmethyl)-N-(C₁₋₃-alkyl)-aminocarbonyl, N-(carboxymethyl)-N-(C₁₋₃-alkyl)-aminocarbonyl or a 4- to 7-membered cycloalkyleneimino-carbonyl group or

(ii) m denotes the number 0,

n denotes the number 0 and

A denotes a —CH₂—CH₂— group, or

(iii) m denotes the number 1,

n denotes the number 0 and

A denotes a —CH₂— group,

the groups R₁ to R₄ are as hereinbefore defined, but R₁ in the 4 position is bound to the phenyl group contained in formula I and

Ar denotes a phenyl group disubstituted by the groups R₅ and R₆, while

R₅ is bound in the 3 position if R₆ denotes a hydrogen atom, or is bound in the 5 position if R₆ assumes a meaning other than the hydrogen atom, and denotes an amidino group optionally substituted by one or two C₁₋₃-alkyl groups, a hydroxy, C₁₋₆-alkoxy-carbonyl, 2,2,2-trichloroethoxycarbonyl or phenylcarbonyl group, or an amino-C₁₋₃-alkyl or C₁₋₃-alkylamino-C₁₋₃-alkyl group and

R₆ denotes a hydrogen atom or a hydroxy, C₁₋₃-alkoxy, carboxy-C₁₋₃-alkoxy, C₁₋₃-alkoxy-carbonyloxy- or C₁₋₄-alkoxy-carbonyl-C₁₋₃-alkoxy group bound in the 2 position,

the stereoisomers and the salts thereof.

Particularly preferred compounds of general formula I are those wherein

(i) m denotes the number 0,

n denotes the number 1 and

A denotes a methylene group wherein

a hydrogen atom may be replaced by a methyl, hydroxycarbonyl, C₁₋₃-alkoxy-carbonyl, C₁₋₃-alkylaminocarbonyl, dimethylaminocarbonyl, pyrrolidin-1-yl-carbonyl, C₁₋₃-alkylaminocarbonylmethyl, N-(hydroxy-carbonyl-methyl)-N-(C₁₋₃-alkyl)-amino-carbonyl-methyl, N-(C₁₋₃-alkoxy-carbonyl-methyl)-N-(C₁₋₃-alkyl)-amino-carbonyl-methyl, hydroxycarbonylmethyl, C₁₋₃-alkoxy-carbonylmethyl or dimethylaminocarbonylmethyl group,

R₁ is bound in the 4 position of the phenyl group of formula I and denotes

a C₅₋₇-cycloalkylamino group which may be substituted at the amino nitrogen atom by a C₁₋₃-alkyl, C₁₋₃-alkylcarbonyl, amino-C₁₋₃-alkylcarbonyl, carboxy-C₁₋₃-alkylcarbonyl or C₁₋₄-alkoxy-carbonyl-C₁₋₃-alkyl-carbonyl group,

a 4- to 7-membered cycloalkyleneiminocarbonyl group

or a 2,5-dihydropyrrol-1-yl-carbonyl group,

R₂ denotes a hydrogen atom or a C₁₋₃-alkyl, ethenyl, ethynyl, trifluoromethyl or trifluoromethoxy group bound in the 3 position or, if R₃ denotes a C₁₋₃-alkyl group, in the 5 position of the phenyl group in formula I or a chlorine or bromine atom bound in the 3 position,

R₃ denotes a hydrogen atom or a C₁₋₃-alkyl group bound in the 2 position of the phenyl group in formula I,

R₄ denotes a hydrogen atom and

Ar denotes a phenyl group disubstituted by the groups R₅ and R₆, while

R₅ is bound in the 3 position if R₆ denotes a hydrogen atom, or is bound in the 5 position if R₆ assumes a meaning other than the hydrogen atom, and denotes an amidino group optionally substituted by a C₁₋₆-alkoxy-carbonyl, 2,2,2-trichloroethoxycarbonyl or phenylcarbonyl group, or a aminomethyl group and

R₆ denotes a hydrogen atom or a hydroxy or C₁₋₃-alkoxy-carbonyloxy group bound in the 2 position,

as well as those compounds wherein

(i) m denotes the number 0,

n denotes the number 0 and

A denotes a —CH₂—CH₂— group, or

(ii) m denotes the number 1,

n denotes the number 0 and

A denotes a —CH₂— group,

R₁ denotes a 4- to 7-membered cycloalkyleneiminocarbonyl or 2,5-dihydropyrrol-1-yl-carbonyl group bound in the 4 position of the phenyl group of formula I,

R₂ denotes a hydrogen atom or a substituent selected from fluorine, chlorine, bromine, C₁₋₃-alkyl and trifluoromethyl bound in the 3 position or, if R₃ denotes a C₁₋₃-alkyl group, bound in the 5 position of the phenyl group in formula I,

R₃ denotes a hydrogen atom or a C₁₋₃-alkyl group bound in the 2 position of the phenyl group in formula I,

R₄ denotes a hydrogen atom and

Ar denotes a phenyl group disubstituted by the groups R₅ and R₆, wherein

R₅ is bound in the 5 position and denotes an amidino group optionally substituted by one or two C₁₋₃-alkyl groups, a C₁₋₆-alkoxy-carbonyl or phenylcarbonyl group and

R₆ denotes a hydroxy group bound in the 2 position,

the stereoisomers and the salts thereof.

The following preferred compounds are mentioned by way of example:

(1) 2-(5-carbamimidoyl-2-hydroxy-phenyl)-N-[3-methyl-4-(pyrrolidin-1-yl-carbonyl)-phenyl]-ethylamine,

(2) N-(5-carbamimidoyl-2-hydroxy-benzyl)-3-methyl-4-(pyrrolidin-1-yl-carbonyl)-benzylamine,

(3) N-(5-carbamimidoyl-2-hydroxy-benzyl)-2,5-dimethyl-4-(pyrrolidin-1-yl-carbonyl)-benzylamine,

(4) N-(5-carbamimidoyl-2-hydroxy-benzyl)-3-methyl-4-(pyrrolidin-1-yl-carbonyl)-benzamide,

(5) N-(5-carbamimidoyl-2-hydroxy-benzyl)-2,5-dimethyl-4-(pyrrolidin-1-yl-carbonyl)-benzamide,

(6) N-(3-carbamimidoyl-benzyl)-3-methyl-4-(pyrrolidin-1-yl-carbonyl)-benzamide,

(7) N-(5-carbamimidoyl-2-hydroxy-benzyl)-3-trifluoromethyl-4-(pyrrolidin-1-yl-carbonyl)-benzamide,

(8) N-(5-aminomethyl-2-hydroxy-benzyl)-3-methyl-4-(pyrrolidin-1-yl-carbonyl)-benzamide,

(9) 2-(3-aminomethyl-phenyl)-2-[3-methyl-4-(pyrrolidin-1-yl-carbonyl)-phenyl]-acetic acid-N-ethylamide,

(10) 3-(3-aminomethyl-phenyl)-3-[3-methyl-4-(pyrrolidin-1-yl-carbonyl)-benzoylamino]-propionic acid-N-ethylamide,

(11) N-(5-carbamimidoyl-2-hydroxy-benzyl)-3-methyl-4-[N-cyclopentyl-N-(3-ethoxy-carbonyl-propionyl)amino]-benzamide,

(12) N-(5-carbamimidoyl-2-hydroxy-benzyl)-3-methyl-4-(N-acetyl-N-cyclobutylamino)-benzamide,

(13) N-(5-carbamimidoyl-2-hydroxy-benzyl)-3-methyl-4-(N-cyclopentyl-N-methyl-amino)-benzamide,

(14) N-(5-carbamimidoyl-2-hydroxy-benzyl)-3-methyl-4-[N-cyclopentyl-N-(3-carboxy-propionyl)amino]-benzamide,

(15) N-(5-carbamimidoyl-2-hydroxy-benzyl)-4-cyclopentylamino-3-methyl-benzamide,

(16) ethyl 2-(3-carbamimidoyl-phenyl)-2-[3-methyl-4-(pyrrolidin-1-yl-carbonyl)-benzoyl-amino]-acetate,

(17) 2-(3-carbamimidoyl-phenyl)-2-[3-methyl-4-(pyrrolidin-1-yl-carbonyl)-benzoyl-amino]-acetic acid,

(18) N-(5-carbamimidoyl-2-hydroxy-benzyl)-3-methyl-4-[N-(2-aminoacetyl)-N-cyclopentyl-amino]-benzamide,

(19) N-(5-carbamimidoyl-2-hydroxy-benzyl)-3-methyl-4-[N-(3-amino-propionyl)-N-cyclopentyl-amino]-benzamide,

(20) N-(5-carbamimidoyl-2-hydroxy-benzyl)-3-chloro-4-(pyrrolidin-1-yl-carbonyl)-benzamide,

(21) ethyl 3-(3-carbamimidoyl-phenyl)-3-[3-methyl-4-(pyrrolidin-1-yl-carbonyl)-benzoyl-amino]-propionate,

(22) ethyl 3-(3-carbamimidoyl-phenyl)-3-[3-chloro-4-(pyrrolidin-1-yl-carbonyl)-benzoyl-amino]-propionate,

(23) ethyl 3-(3-carbamimidoyl-phenyl)-3-{3-methyl-4-[N-(3-amino-propionyl)-N-cyclo-pentyl-amino]-benzoylamino}-propionate,

(24) ethyl 3-(3-carbamimidoyl-phenyl)-3-[3-bromo-4-(pyrrolidin-1-yl-carbonyl)-benzoyl-amino]-propionate,

(25) ethyl 3-(3-carbamimidoyl-phenyl)-3-[3-methyl-4-(2,5-dihydropyrrol-1-yl-carbonyl)-benzoylamino]-propionate,

(26) ethyl 3-(3-carbamimidoyl-phenyl)-3-[3-ethynyl-4-(pyrrolidin-1-yl-carbonyl)-benzoyl-amino]-propionate,

(27) ethyl 3-(3-carbamimidoyl-phenyl)-3-[3-ethyl-4-(pyrrolidin-1- yl-carbonyl)-benzoyl-amino]-propionate,

(28) ethyl 3-(3-carbamimidoyl-phenyl)-3-[3-ethenyl-4-(pyrrolidin-1-yl-carbonyl)-benzoyl-amino]-propionate,

(29) 3-(3-carbamimidoyl-phenyl)-3-[3-methyl-4-(pyrrolidin-1-yl-carbonyl)-benzoyl-amino]-propionic acid,

(30) 3-(3-carbamimidoyl-phenyl)-3-[3-trifluoromethyl-4-(pyrrolidin-1-yl-carbonyl)-benzoylamino]-propionic acid,

(31) 3-(3-carbamimidoyl-phenyl)-3-[3-chloro-4-(pyrrolidin-1-yl-carbonyl)-benzoylamino]-propionic acid,

(32) 3-(3-carbamimidoyl-phenyl)-3-[3-methyl-4-(2,5-dihydropyrrol-1-yl-carbonyl)-benzoylamino]-propionic acid,

(33) 3-(3-carbamimidoyl-phenyl)-3-[3-ethynyl-4-(pyrrolidin-1-yl-carbonyl)-benzoyl-amino]-propionic acid,

(34) 3-(3-carbamimidoyl-phenyl)-3-[3-ethyl-4-(pyrrolidin-1-yl-carbonyl)-benzoylamino]-propionic acid,

(35) 3-[3-methyl-4-(pyrrolidin-1-yl-carbonyl)-benzoylamino]-3-(3-carbamimidoyl-phenyl)-propionic acid-N-methyl-N-(hydroxycarbonylmethyl)-amide,

(36) 3-[3-methyl-4-(pyrrolidin-1-yl-carbonyl)-benzoylamino]-3-(3-carbamimidoyl-phenyl)-propionic acid-N-(hydroxycarbonylmethyl)-N-(n-propyl)-amide,

(37) 3-(3-carbamimidoyl-phenyl)-3-[3-ethenyl-4-(pyrrolidin-1-yl-carbonyl)-benzoyl-amino]-propionic acid,

(38) 3-(3-carbamimidoyl-phenyl)-3-[3-methyl-4-(pyrrolidin-1-yl-carbonyl)-benzoyl-amino]-propionic acid-N,N-dimethylamide,

(39) N-[1-(3-carbamimidoyl-phenyl)-2-oxo-2-(pyrrolidin-1-yl)-ethyl]-3-methyl-4-(pyrrolidin-1-yl-carbonyl)-benzamide,

(40) 2-(3-carbamimidoyl-phenyl)-2-[3-methyl-4-(pyrrolidin-1-yl-carbonyl)-benzoyl-amino]-acetic acid-N,N-dimethylamide,

(41) 2-(3-carbamimidoyl-phenyl)-2-[3-methyl-4-(pyrrolidin-1-yl-carbonyl)-benzoyl-amino]-acetic acid-N-ethylamide,

(42) 3-(3-carbamimidoyl-phenyl)-3-[3-methyl-4-(pyrrolidin-1-yl-carbonyl)-benzoyl-amino]-propionic acid-N-ethylamide,

(43) 3-[3-methyl-4-(pyrrolidin-1-yl-carbonyl)-benzoylamino]-3-(3-carbamimidoyl-phenyl)-propionic acid-N-(ethoxycarbonylmethyl)-N-(n-propyl)-amide,

(44) N-[1-(5-carbamimidoyl-2-hydroxy-phenyl)-ethyl]-3-methyl-4-(pyrrolidin-1-yl-carbonyl)-benzamide,

(45) N-[1-(5-carbamimidoyl-2-hydroxy-phenyl)-ethyl]-3-bromo-4-(pyrrolidin-1-yl-carbonyl)-benzamide,

(46) N-[1-(5-carbamimidoyl-2-hydroxy-phenyl)-ethyl]-4-(pyrrolidin-1-yl-carbonyl)-benzamide,

(47) ethyl 3-(3-carbamimidoyl-phenyl)-3-[3-trifluoromethyl-4-(pyrrolidin-1-yl-carbonyl)-benzoylamino]-propionate,

(48) N-(5-carbamimidoyl-2-hydroxy-benzyl)-3-trifluoromethoxy-4-(pyrrolidin-1-yl-carbonyl)-benzamide,

(49) 3-(5-carbamimidoyl-2-hydroxy-phenyl)-3-[3-methyl-4-(pyrrolidin-1-yl-carbonyl)-benzoylamino]-propionic acid,

(50) ethyl 3-[3-N-(phenylcarbonyl)-amidino-phenyl]-3-[3-methyl-4-(pyrrolidin-1-yl-carbonyl)-benzoylamino]-propionate,

(51) ethyl 3-[3-N-(n-hexyloxycarbonyl)-amidino-phenyl]-3-[3-methyl-4-(pyrrolidin-1-yl-carbonyl)-benzoylamino]-propionate,

(52) n-propyl 3-[3-N-(phenylcarbonyl)-amidino-phenyl]-3-[3-methyl-4-(pyrrolidin-1-yl-carbonyl)-benzoylamino]-propionate,

(53) ethyl 3-[3-N-(2,2,2-trichloroethyloxycarbonyl)-amidino-phenyl]-3-[3-methyl-4-(pyrrolidin-1-yl-carbonyl)-benzoylamino]-propionate,

(54) N-{5-[N-(n-hexyloxycarbonyl)-amidino]-2-hydroxy-benzyl}-3-methyl-4-(pyrrolidin-1-yl-carbonyl)-benzamide,

(55) N-{5-[N-(phenylcarbonyl)-amidino]-2-hydroxy-benzyl}-3-methyl-4-(pyrrolidin-1-yl-carbonyl)-benzamide,

(56) N-[5-(N-hydroxy-amidino)-2-hydroxy-benzyl]-3-methyl-4-(pyrrolidin-1-yl-carbonyl)-benzamide and

(57) N-{5-[N-(phenylcarbonyl)-amidino]-2-(ethyloxycarbonyloxy)-benzyl}-3-methyl-4-(pyrrolidin-1-yl-carbonyl)-benzamide,

wherein any amidino group present may additionally be substituted by a C₁₋₆-alkoxy-carbonyl or phenylcarbonyl group, and the salts thereof.

According to the invention, the compounds of general formula I are obtained by methods known per se, e.g. by the following processes:

a) In order to prepare a compound of general formula I wherein

(i) m denotes the number 0, n denotes the number 1 and A denotes a straight-chain C₁₋₃-alkylene group wherein

one or two hydrogen atoms independently of one another may be replaced in each case by a C₁₋₃-alkyl group or

a hydrogen atom may be replaced by the group —(CH₂)ₚ—R_{f}, while p and R_{f} are as hereinbefore defined, or

(ii) m and n each denote the number 1 and A denotes a bond and

Ar denotes a phenyl or naphthyl group substituted by the groups R₅, R₆ and R₇, while R₆ and R₇ are as hereinbefore defined and R₅ denotes an amidino group:

acylating a compound of general formula

H—NR₄—A—Ar (II),

wherein R₄ is as hereinbefore defined,

A denotes a straight-chain C₁₋₃-alkylene group wherein one or two hydrogen atoms independently of one another may be replaced in each case by a C₁₋₃-alkyl group or a hydrogen atom may be replaced by the group —(CH₂)ₚ—R_{f}, while p and R_{f} are as hereinbefore defined, or denotes a bond and

Ar denotes a phenyl or naphthyl group substituted by the groups R₅, R₆ and R₇, while R₅ denotes a cyano group and R₆ and R₇ are as hereinbefore defined,

with a carboxylic acid or a reactive carboxylic acid derivative of general formula

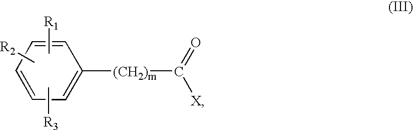

wherein m denotes the number 0 or 1, X denotes a hydroxy or C₁₋₄-alkoxy group or a chlorine atom and R₁ to R₃ are as hereinbefore defined, or with the reactive derivatives thereof and subsequently converting the cyano compound thus obtained into an amidino compound.

The acylation is conveniently carried out with a corresponding halide or anhydride in a solvent such as methylene chloride, chloroform, carbon tetrachloride, ether, tetrahydrofuran, dioxan, benzene, toluene, acetonitrile, dimethylformamide, sodium hydroxide solution or sulpholane optionally in the presence of an inorganic or organic base at temperatures between −20 and 200° C., but preferably at temperatures between −10 and 160° C.

The acylation may however also be carried out with the free acid optionally in the presence of an acid-activating agent or a dehydrating agent, e.g. in the presence of isobutyl chloroformate, thionyl chloride, trimethylchlorosilane, hydrogen chloride, sulphuric acid, methanesulphonic acid, p-toluenesulphonic acid, phosphorus trichloride, phosphorus pentoxide, N,N′-dicyclohexylcarbodiimide, N,N′-dicyclohexyl-carbodiimide/N-hydroxysuccinimide or 1-hydroxy-benzotriazole, N,N′-carbonyldiimidazole, O-(benzotriazol-1-yl)-N,N,N′,N′-tetramethyl-uronium tetrafluoroborate/N-methylmorpholine, O-(benzotriazol-1-yl)-N,N,N′,N′-tetramethyl-uronium tetrafluoroborate/N-ethyldiisopropylamine, N,N′-thionyldiimidazole or triphenylphosphine/carbon tetrachloride, at temperatures between −20 and 200° C., but preferably at temperatures between −10 and 160° C.

The subsequent conversion of the cyano group into an amidino group takes place as described in process e).

b) In order to prepare a compound of general formula I wherein m denotes the number 0 or

n denotes the number 0,

A denotes a straight-chain C₁₋₃-alkylene group wherein one or two hydrogen atoms independently of one another may be replaced in each case by a C₁₋₃-alkyl group, and

Ar denotes a phenyl or naphthyl group substituted by the groups R₅, R₆ and R₇, while R₆ and R₇ are as hereinbefore defined and R₅ denotes an amidino group:

alkylating a compound of general formula

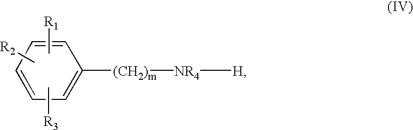

wherein R₁ to R₄ are as hereinbefore defined and m denotes the number 0 or 1, with a compound of general formula

Z₁—A—Ar (V),

wherein A denotes a straight-chain C₁₋₃-alkylene group wherein one or two hydrogen atoms independently of one another may be replaced in each case by a C₁₋₃-alkyl group,

Ar denotes a phenyl or naphthyl group substituted by the groups R₅, R₆ and R₇, while R₆ and R₇ are as hereinbefore defined and R₅ denotes a cyano group,

and Z₁ denotes a leaving group such as a halogen atom or a sulphonyloxy group, e.g. a chlorine, bromine or iodine atom or a trifluoromethylsulphonyloxy group, and subsequently converting the cyano compound thus obtained into an amidino compound.

The alkylation is optionally carried out in a solvent or mixture of solvents such as methylene chloride, dimethylformamide, benzene, toluene, chlorobenzene, tetra-hydrofuran, benzene/tetrahydrofuran, dioxan, dimethylsulphoxide or sulpholane with an alkylating agent such as a corresponding halide or sulphonic acid ester, e.g. with methyl iodide, ethyl bromide, dimethylsulphate or benzyl chloride, optionally in the presence of a tertiary organic base or in the presence of an inorganic base, conveniently at temperatures between 0 and 150° C., preferably at temperatures between 0 and 100° C.

The subsequent conversion of the cyano group into an amidino group is carried out as described in process e).

c) In order to prepare a compound of general formula I wherein

Ar denotes a phenyl or naphthyl group substituted by the groups R₅, R₆ and R₇, while R₆ and R₇ are as hereinbefore defined and R₅ denotes an amidino group,

m denotes the number 1, n denotes the number 0 and

A denotes a straight-chain C₁₋₃-alkylene group wherein one or two hydrogen atoms independently of one another may be replaced in each case by a C₁₋₃-alkyl group, or m denotes the number 2, n denotes the number 0 and A denotes a bond:

alkylating a compound of general formula

HNR₄ —A—Ar (II′),

wherein R₄ is as hereinbefore defined,

A denotes a straight-chain C₁₋₃-alkylene group wherein one or two hydrogen atoms independently of one another may be replaced in each case by a C₁₋₃-alkyl group, or denotes a bond, and

Ar denotes a phenyl or naphthyl group substituted by the groups R₅, R₆ and R₇, while R₆ and R₇ are as hereinbefore defined and R₅ denotes a cyano group,

with a compound of general formula

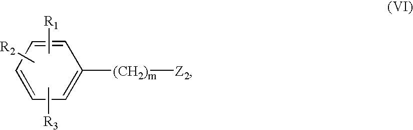

wherein R₁ to R₃ are as hereinbefore defined, m denotes the number 1 or 2 and Z₂ denotes a leaving group such as a halogen atom or a sulphonyloxy group, e.g. a chlorine, bromine or iodine atom or a trifluoromethylsulphonyloxy group, and subsequently converting the resulting cyano compound into an amidino compound.

The alkylation is optionally carried out in a solvent or mixture of solvents such as methylene chloride, dimethylformamide, benzene, toluene, chlorobenzene, tetrahydrofuran, benzene/tetrahydrofuran, dioxan, dimethylsulphoxide or sulpholane with an alkylating agent such as a corresponding halide or sulphonic acid ester, e.g. with methyl iodide, ethyl bromide, dimethylsulphate or benzyl chloride, optionally in the presence of a tertiary organic base or in the presence of an inorganic base conveniently at temperatures between 0 and 150° C., preferably at temperatures between 0 and 100° C.

The subsequent conversion of the cyano group into an amidino group is carried out as described in process e).

d) In order to prepare a compound of general formula I wherein

Ar denotes a phenyl or naphthyl group substituted by the groups R₅, R₆ and R₇, while R₆ and R₇ are as hereinbefore defined and R₅ denotes an amidino group,

m denotes the number 0 or 1, n denotes the number 0 and

A denotes a straight-chain C₁₋₃-alkylene group wherein one or two hydrogen atoms independently of one another may be replaced in each case by a C₁₋₃-alkyl group, or

m denotes the number 2, n denotes the number 0 and A denotes a bond: reductive alkylation of an amine of general formula

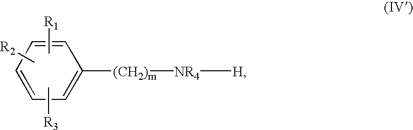

wherein R₁ to R₄ are as hereinbefore defined and m denotes the number 0, 1 or 2, with an aldehyde of general formula

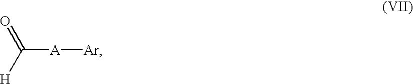

wherein A denotes a straight-chain C₁₋₃-alkylene group wherein one or two hydrogen atoms independently of one another may be replaced in each case by a C₁₋₃-alkyl group, or denotes a bond, and

Ar denotes a phenyl or naphthyl group substituted by the groups R₅, R₆ and R₇, while R₆ and R₇ are as hereinbefore defined and R₅ denotes a cyano group, and subsequently converting the resulting cyano compound into an amidino compound.

The reductive alkylation is however preferably carried out in the presence of a complex metal hydride such as sodium borohydride, lithium borohydride, sodium cyanoborohydride, zinc borohydride, sodium triacetoxyborohydride or borane/pyridine conveniently at a pH of 1-7 optionally in the presence of a dehydrating agent such as molecular sieve or titanium-IV-isopropoxide and at ambient temperature or with hydrogen in the presence of a hydrogenation catalyst, e.g. in the presence of palladium/charcoal, at a hydrogen pressure of 1 to 5 bar, preferably at temperatures between 20° C. and the boiling temperature of the solvent used. It may also be advantageous during the reaction if reactive groups are protected during the reaction by conventional protecting groups which are cleaved again by conventional methods after the reaction.

The subsequent conversion of the cyano group into an amidino group is carried out as described in process e).

e) In order to prepare a compound of general formula I wherein Ar denotes a phenyl or naphthyl group substituted by the groups R₅, R₆ and R₇, while R₆ and R₇ are as hereinbefore defined and R₅ denotes an amidino group optionally substituted by one or two C₁₋₃-alkyl groups:

reacting a compound of general formula

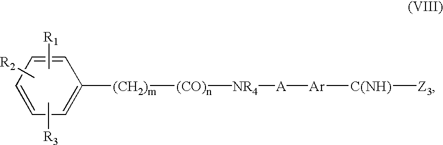

optionally formed in the reaction mixture,

wherein

R₁ to R₄, m, n and A are as hereinbefore defined, Ar denotes a phenyl or naphthyl group substituted by the groups R₆ and R₇, while R₆ and R₇ are as hereinbefore defined, and Z₃ denotes an alkoxy or aralkoxy group such as the methoxy, ethoxy, n-propoxy, iso-propoxy or benzyloxy group or an alkylthio or aralkylthio group such as the methylthio, ethylthio, n-propylthio or benzylthio group, with an amine of general formula

H—R₈NR₉, (IX)

wherein

R₈ and R₉, which may be identical or different, each denote a hydrogen atom or a C₁₋₃-alkyl group, or with the salts thereof.

The reaction is conveniently carried out in a solvent such as methanol, ethanol, n-propanol, tetrahydrofuran or dioxan at temperatures between 0 and 150° C., preferably at temperatures between 0 and 80° C., with an amine of general formula IX or with a corresponding acid addition salt such as for example ammonium carbonate or ammonium acetate.

A compound of general formula VIII is obtained for example by reacting a corresponding cyano compound with a corresponding alcohol such as methanol, ethanol, n-propanol, isopropanol or benzyl alcohol in the presence of an acid such as hydrochloric acid or by reacting a corresponding amide with a trialkyloxonium salt such as triethyloxonium-tetrafluoroborate in a solvent such as methylene chloride, tetrahydrofuran or dioxan at temperatures between 0 and 50° C., but preferably at 20° C., or a corresponding nitrile with hydrogen sulphide conveniently in a solvent such as pyridine or dimethylformamide and in the presence of a base such as triethylamine and subsequently alkylating the thioamide formed with a corresponding alkyl or aralkyl halide.

f) In order to prepare a compound of general formula I wherein Ar denotes a phenyl or naphthyl group substituted by the groups R₅, R₆ and R₇, while R₆ and R₇ are as hereinbefore defined and R₅ denotes an aminomethyl, C₁₋₃-alkylaminomethyl or di-(C₁₋₃-alkyl)aminomethyl group:

Catalytic hydrogenation of a compound of general formula

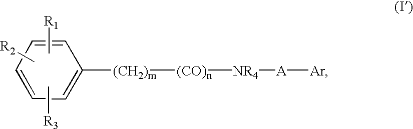

wherein

Ar denotes a phenyl or naphthyl group substituted by the groups R₅, R₆ and R₇, R₁ to R₄, R₆, R₇, A, m and n are as hereinbefore defined and R₅ denotes a cyano group, and optionally subsequent alkylation with a compound of formula

R₁₀—Z₄ (X),

wherein R₁₀ denotes a C₁₋₃-alkyl group and Z₄ denotes a leaving group such as a halogen atom or a sulphonyloxy group, e.g. a chlorine, bromine or iodine atom or a trifluoromethyl-sulphonyloxy group.

The catalytic hydrogenation is carried out with hydrogen in the presence of a catalyst such as palladium/charcoal, platinum in a solvent such as methanol, ethanol, ethyl acetate, dimethylformamide, dimethylformamide/acetone or glacial acetic acid optionally with the addition of an acid such as hydrochloric acid at temperatures between 0 and 50° C., but preferably at ambient temperature, and at a hydrogen pressure of 1 to 7 bar, but preferably 3 to 5 bar, or for example with Raney nickel preferably in methanolic ammonia solution.

The alkylation which optionally follows is conveniently carried out in a solvent or mixture of solvents such as methylene chloride, dimethylformamide, benzene, toluene, chlorobenzene, tetrahydrofuran, benzene/tetrahydrofuran, dioxan, dimethylsulphoxide or sulpholane with an alkylating agent such as a corresponding halide or sulphonic acid ester, e.g. with methyl iodide, ethyl bromide, dimethylsulphate or benzyl chloride, optionally in the presence of a tertiary organic base or in the presence of an inorganic base conveniently at temperatures between 0 and 150° C., preferably at temperatures between 0 and 100° C.

g) In order to prepare a compound of general formula I wherein

m denotes the number 0, n denotes the number 0, A denotes a straight-chain C₁₋₃-alkylene group wherein one or two hydrogen atoms independently of one another may be replaced in each case by a C₁₋₃-alkyl group, and

Ar denotes a phenyl or naphthyl group substituted by the groups R₅, R₆ and R₇, while R₆ and R₇ are as hereinbefore defined and R₅ denotes an amidino group:

coupling a compound of general formula

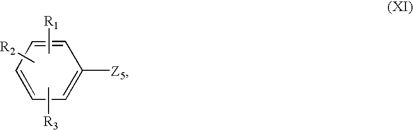

wherein

R₁ to R₃ are as hereinbefore defined and Z₅ denotes a leaving group such as a halogen atom or a sulphonyloxy group, e.g. a chlorine, bromine or iodine atom or a trifluoromethylsulphonyloxy group,

with a compound of general formula

HNR₄ —A—Ar (II″),

wherein R₄ is as hereinbefore defined, A denotes a straight-chain C₁₋₃-alkylene group wherein one or two hydrogen atoms independently of one another may be replaced in each case by a C₁₋₃-alkyl group, and

Ar denotes a phenyl or naphthyl group substituted by the groups R₅, R₆ and R₇, while R₆ and R₇ are as hereinbefore defined and R₅ denotes a cyano group, and subsequently converting the resulting cyano compound into an amidino compound.

The coupling reaction is conveniently carried out in a solvent such as toluene, dioxan, dimethoxyethane or tetrahydrofuran using a suitable catalyst, for example bis-(tri-o-tolylphosphine)-palladium-(II)-chloride, tris-(dibenzylideneacetone)-dipalladium(0)/tris-o-tolylphosphine, tris-(dibenzylideneacetone)-dipalladium(0)/tris-(2-furyl)phosphan, tris-(dibenzylideneacetone)-dipalladium(1)/2,2′-bis-(diphenylphosphino)-1,1′-binaphthyl, tetrakis-(triphenylphosphine)-palladium(0), 1,1 ′-bis-(diphenylphosphino)-ferrocene-palladium-dichloride or palladium-II-acetate/1,3-bis-(triphenylphosphino)-propane, preferably in the presence of a base such as sodium-tert.butoxide, bis-(trimethylsilyl)-lithium amide, potassium carbonate, caesium carbonate or triethylamine at a temperature between 0 and 150° C., preferably 20 to 100° C.

The subsequent conversion of the cyano group into an amidino group is carried out as described in process e).

If according to the invention a compound of general formula I is obtained which contains an amino or imino group, this may subsequently be converted with a corresponding acyl derivative into a corresponding acyl compound of general formula I and/or

if a compound of general formula I is obtained which contains an esterified carboxy group, this may be converted by hydrolysis into a corresponding carboxylic acid of general formula I and/or

if a compound of general formula I is obtained which contains a carboxy group, this may subsequently be converted by esterification into a corresponding ester.

The subsequent acylation is conveniently carried out with a corresponding halide or anhydride in a solvent such as methylene chloride, chloroform, carbon tetrachloride, ether, tetrahydrofuran, dioxan, benzene, toluene, acetonitrile or sulpholane optionally in the presence of an inorganic or organic base at temperatures between −20 and 200° C., but preferably at temperatures between −10 and 160° C. This may however also be carried out with the free acid, optionally in the presence of an acid-activating agent or a dehydrating agent, e.g. in the presence of isobutyl chloroformate, thionylchloride, trimethylchlorosilane, hydrogen chloride, sulphuric acid, methanesulphonic acid, p-toluenesulphonic acid, phosphorus trichloride, phosphorus pentoxide, N,N′-dicyclohexylcarbodiimide, N,N′-dicyclohexylcarbodiimide/N-hydroxysuccinimide or 1-hydroxy-benzotriazole, N,N′-carbonyldiimidazole or N,N′-thionyldiimidazole or triphenylphosphine/carbon tetrachloride, at temperatures between −20 and 200° C., but preferably at temperatures between −10 and 160° C.

The subsequent hydrolysis is conveniently carried out either in the presence of an acid such as hydrochloric acid, sulphuric acid, phosphoric acid, acetic acid, trichloroacetic acid, trifluoroacetic acid or mixtures thereof or in the presence of a base such as lithium hydroxide, sodium hydroxide or potassium hydroxide in a suitable solvent such as water, water/methanol, water/ethanol, water/isopropanol, methanol, ethanol, water/tetrahydrofuran or water/dioxan and the subsequent decarboxylation in the presence of an acid as hereinbefore described at temperatures between −10 and 120° C., e.g. at temperatures between ambient temperature and the boiling temperature of the reaction mixture.

The subsequent esterification is carried out with a corresponding alcohol, conveniently in a solvent or mixture of solvents such as methylene chloride, benzene, toluene, chlorobenzene, tetrahydrofuran, benzene/tetrahydrofuran or dioxan, but preferably in an excess of the alcohol used, optionally in the presence of an acid such as hydrochloric acid or in the presence of a dehydrating agent, e.g. in the presence of isobutyl chloroformate, thionyl chloride, trimethylchlorosilane, hydrochloric acid, sulphuric acid, methanesulphonic acid, p-toluenesulphonic acid, phosphorus trichloride, phosphorus pentoxide, N,N′-dicyclohexylcarbodiimide, N,N′-dicyclohexylcarbodiimide/N-hydroxysuccinimide, N,N′-carbonyldiimidazole or N,N′-thionyldiimidazole, triphenylphosphine/carbon tetrachloride or triphenyl-phosphine/diethyl azodicarboxylate, optionally in the presence of a base such as potassium carbonate, N-ethyl-diisopropylamine or N,N-dimethylamino-pyridine, conveniently at temperatures between 0 and 150° C., preferably at temperatures between 0 and 80° C., or with a corresponding halide in a solvent such as methylene chloride, tetrahydrofuran, dioxan, dimethylsulphoxide, dimethylformamide or acetone optionally in the presence of a reaction accelerator such as sodium or potassium iodide and preferably in the presence of a base such as sodium carbonate or potassium carbonate or in the presence of a tertiary organic base such as N-ethyl-diisopropylamine or N-methyl-morpholine, which may also simultaneously serve as the solvent, or optionally in the presence of silver carbonate or silver oxide at temperatures between −30 and 100° C., but preferably at temperatures between −10 and 80° C.

In the reactions described hereinbefore, any reactive groups present such as hydroxy, carboxy, amino, alkylamino or imino groups may be protected during the reaction by conventional protecting groups which are cleaved again after the reaction.

For example, a protecting group for a hydroxy group may be a methoxy, benzyloxy, trimethylsilyl, acetyl, benzoyl, tert-butyl, trityl, benzyl or tetrahydropyranyl group,

protecting groups for a carboxy group may be a trimethylsilyl, methyl, ethyl, tert.butyl, benzyl or tetrahydropyranyl group and

protecting groups for an amino, alkylamino or imino group may be an acetyl, trifluoroacetyl, benzoyl, ethoxycarbonyl, tert-butoxycarbonyl, benzyloxycarbonyl, benzyl, methoxybenzyl or 2,4-dimethoxybenzyl group and additionally, for the amino group, a phthalyl group.

Any protecting group used is optionally subsequently cleaved for example by hydrolysis in an aqueous solvent, e.g. in water, isopropanol/water, acetic acid/water, tetrahydrofuran/water or dioxan/water, in the presence of an acid such as trifluoroacetic acid, hydrochloric acid or sulphuric acid or in the presence of an alkali metal base such as lithium hydroxide, sodium hydroxide or potassium hydroxide or by ether splitting, e.g. in the presence of iodotrimethylsilane, at temperatures between 0 and 100° C., preferably at temperatures between 10 and 50° C.

However, a benzyl, methoxybenzyl or benzyloxycarbonyl group is cleaved, for example, hydrogenolytically, e.g. with hydrogen in the presence of a catalyst such as palladium/charcoal in a solvent such as methanol, ethanol, ethyl acetate, dimethylformamide, dimethylformamide/acetone or glacial acetic acid optionally with the addition of an acid such as hydrochloric acid at temperatures between 0 and 50° C., but preferably at ambient temperature, and at a hydrogen pressure of 1 to 7 bar, but preferably 3 to 5 bar.

A methoxybenzyl group may also be cleaved in the presence of a oxidising agent such as cerium(IV)ammonium nitrate in a solvent such as methylene chloride, acetonitrile or acetonitrile/water at temperatures between 0 and 50° C., but preferably at ambient temperature.

A methoxy group is conveniently cleaved in the presence of boron tribromide in a solvent such as methylene chloride at temperatures between −35 and −25° C.

A 2,4-dimethoxybenzyl group, however, is preferably cleaved in trifluoroacetic acid in the presence of anisole.

A tert.butyl or tert.butyloxycarbonyl group is preferably cleaved by treating with an acid such as trifluoroacetic acid or hydrochloric acid, optionally using a solvent such as methylene chloride, dioxan or ether.

A phthalyl group is preferably cleaved in the presence of hydrazine or a primary amine such as methylamine, ethylamine or n-butylamine in a solvent such as methanol, ethanol, isopropanol, toluene/water or dioxan at temperatures between 20 and 50° C.

An allyloxycarbonyl group is cleaved by treating with a catalytic amount of tetrakis-(triphenylphosphine)-palladium(O), preferably in a solvent such as tetrahydrofuran and preferably in the presence of an excess of a base such as morpholine or 1,3-dimedone at temperatures between 0 and 100° C., preferably at ambient temperature and under inert gas, or by treating with a catalytic amount of tris-(triphenylphosphine)-rhodium(I)chloride in a solvent such as aqueous ethanol and optionally in the presence of a base such as 1,4-diazabicyclo[2.2.2]octane at temperatures between 20 and 70° C.

The compounds of general formulae II to XI used as starting materials, some of which are known from the literature, are obtained by methods known from the literature and their preparation is also described in the Examples.

The chemistry of the compounds of general formula II, II′, II″ IV and IV′ is described, for example, by Schröter in Stickstoffverbindungen II, pages 341-730, Methoden der organischen Chemie (Houben-Weyl), 4^{th} edition, Verlag Thieme, Stuttgart 1957. The preparation of carboxylic acid derivatives of general formula III is described in Methoden der organischen Chemie (Houben-Weyl), Volume E5, Carbonsäuren und Carbonsäurederivate, 4^{th} edition, Verlag Thieme, Stuttgart 1985.

Moreover, the compounds of general formula I obtained may be resolved into their enantiomers and/or diastereomers.

Thus, for example, the compounds of general formula I obtained which occur as racemates may be separated by methods known per se (cf. Allinger N. L. and Eliel E. L. in “Topics in Stereochemistry”, Vol. 6, Wiley Interscience, 1971) into their optical enantiomers and compounds of general formula I with at least 2 asymmetric carbon atoms may be resolved into their diastereomers on the basis of their physical-chemical differences using methods known per se, e.g. by chromatography and/or fractional crystallisation, and, if these compounds are obtained in racemic form, they may subsequently be resolved into the enantiomers as mentioned above.

The enantiomers are preferably separated by column separation on chiral phases or by recrystallisation from an optically active solvent or by reacting with an optically active substance which forms salts or derivatives such as e.g. esters or amides with the racemic compound, particularly acids and the activated derivatives or alcohols thereof, and separating the diastereomeric mixture of salts or derivatives thus obtained, e.g. on the basis of their differences in solubility, whilst the free antipodes may be released from the pure diastereomeric salts or derivatives by the action of suitable agents. Optically active acids in common use are e.g. the D- and L-forms of tartaric acid or dibenzoyltartaric acid, di-o-tolyltartaric acid, malic acid, mandelic acid, camphorsulphonic acid, glutamic acid, aspartic acid or quinic acid. An optically active alcohol may be for example (+) or (−)-menthol and an optically active acyl group in amides, for example, may be a (+)- or (−)-menthyloxycarbonyl.

Furthermore, the compounds of formula I may be converted into the salts thereof, particularly for pharmaceutical use into the physiologically acceptable salts with inorganic or organic acids. Acids which may be used for this purpose include for example hydrochloric acid, hydrobromic acid, sulphuric acid, methanesulphonic acid, phosphoric acid, fumaric acid, succinic acid, lactic acid, citric acid, tartaric acid or maleic acid.

Moreover, if the new compounds of formula I contain a carboxy group, they may subsequently, if desired, be converted into the salts thereof with inorganic or organic bases, particularly for pharmaceutical use into the physiologically acceptable salts thereof. Suitable bases for this purpose include for example sodium hydroxide, potassium hydroxide, cyclohexylamine, ethanolamine, diethanolamine and triethanolamine.

As already mentioned, the new compounds of general formula I and the salts thereof have valuable properties. Thus, the compounds of general formula I wherein Ar denotes a phenyl or naphthyl group substituted by the groups R₅, R₆ and R₇ and R₅ denotes a cyano group are valuable intermediates for preparing the corresponding compounds of general formula I wherein R₅ denotes an amidino group optionally substituted by one or two C₁₋₃-alkyl groups. The compounds of general formula I with the exception of those compounds wherein Ar denotes a phenyl or naphthyl group substituted by the groups R₅, R₆ and R₇ and R₅ denotes a cyano group, as well as the tautomers, the stereoisomers and the physiologically acceptable salts thereof, have valuable pharmacological properties, particularly an antithrombotic activity which is preferably based on an effect on thrombin or factor Xa, on a prolonging effect on aPTT time and on an inhibitory effect on related serine proteases such as e.g. trypsin, urokinase, factor VIIa, factor IX, factor XI and factor XII.

For example, the compounds

(1) 2-(5-carbamimidoyl-2-hydroxy-phenyl)-N-[3-methyl-4-(pyrrolidin-1-yl-carbonyl)-phenyl]-ethylamine-hydrochloride,

(2) N-(5-carbamimidoyl-2-hydroxy-benzyl)-3-methyl-4-(pyrrolidin-1-yl-carbonyl)-benzamide-hydrochloride,

(3) N-(5-carbamimidoyl-2-hydroxy-benzyl)-3-methyl-4-[N-cyclopentyl-(3-ethoxy-carbonylpropionyl)amino]-benzamide-hydrochloride,

(4) N-(5-carbamimidoyl-2-hydroxy-benzyl)-3-chloro-4-(pyrrolidin-1-yl-carbonyl)-benzamide and

(5) 3-(3-carbamimidoyl-phenyl)-3-[3-methyl-4-(pyrrolidin-1-yl-carbonyl)-benzoylamino]-propionic acid,

were investigated for their effect on the inhibition of factor Xa as follows:

Method:

Enzyme-kinetic measurement with chromogenic substrate. The quantity of anp-nitroaniline (pNA) released from the colourless chromogenic substrate by human factor Xa is determined photometrically at 405 nm. It is proportional to the activity of the enzyme used. The inhibition of the enzyme activity by the test substance I (in relation to the solvent control) is determined at various concentrations of test substance and from this the IC₅₀ is calculated, as the concentration which inhibits the factor Xa used by 50%.

Material:

Tris(hydroxymethyl)-aminomethane buffer (100 mmol) and sodium chloride (150 mMol), pH 8.0

Factor Xa (Roche), spec. activity: 10 U/0.5 ml, final concentration: 0.175 U/ml for each reaction mixture

Substrate Chromozym X (Roche), final concentration: 200 μMol/l for each reaction mixture

Test substance: final concentration 100, 30, 10, 3, 1, 0.3, 0.1, 0.03, 0.01, 0.003, 0.001 μMol/l

Procedure:

10 μl of a 23.5-times concentrated starting solution of the test substance or solvent (control), 175 μl of tris(hydroxymethyl)-aminomethane buffer and 25 μl of a 1.65 U/ml Factor Xa working solution are incubated for 10 minutes at 37° C. After the addition of 25 μl of Chromozym X working solution (1.88 μMol/l) the sample is measured in a photometer (SpectraMax 250) at 405 nm for 150 seconds at 37° C.

Evaluation:

1. Determining the maximum increase (deltaOD/minutes) over 3 measuring points.

2. Determining the % inhibition based on the solvent control.

3. Plotting a dosage/activity curve (% inhibition vs substance concentration).

4. Determining the IC₅₀ by interpolating the X value (substance concentration) of the dosage/activity curve at Y=50% inhibition.

The following Table shows the results obtained:

| | Inhibition of factor Xa |
|---|---|
| Substance | (IC₅₀ in μM) |
| (1) | 0.084 |
| (2) | 0.014 |
| (3) | 0.075 |
| (4) | 0.01 |
| (5) | 0.031 |

The compounds prepared according to the invention are well tolerated, as no toxic side effects could be observed at therapeutic doses.

In view of their pharmacological properties the new compounds, with the exception of those compounds wherein Ar denotes a phenyl or naphthyl group substituted by the groups R₅, R₆ and R₇, and R₅ denotes a cyano group, and the physiologically acceptable salts thereof are suitable for the prevention and treatment of venous and arterial thrombotic diseases, such as for example the treatment of deep leg vein thrombosis, for preventing reocclusions after bypass operations or angioplasty (PT(C)A), and occlusion in peripheral arterial diseases such as pulmonary embolism, disseminated intravascular coagulation, for preventing coronary thrombosis, stroke and the occlusion of shunts. In addition, the compounds according to the invention are suitable for antithrombotic support in thrombolytic treatment, such as for example with alteplase, reteplase, tenecteplase, staphylokinase or streptokinase, for preventing long-term restenosis after PT(C)A, for the prevention and treatment of ischaemic incidents in patients with unstable angina or non-transmural cardiac infarct, for preventing metastasis and the growth of clot-dependent tumours and fibrin-dependent inflammatory processes, e.g. in the treatment of pulmonary fibrosis, for the prevention and treatment of rheumatoid arthritis, for preventing fibrin-dependent tissue adhesions and/or the formation of scar tissue and for promoting wound healing processes. The new compounds and the physiologically acceptable salts thereof may be used therapeutically in conjunction with inhibitors of platelet aggregation such as fibrinogen receptor antagonists (e.g. abciximab, eptifibatide, tirofiban), with inhibitors of ADP-induced aggregation (e.g. clopidogrel, ticlopidine), with P₂T receptor antagonists (e.g. cangrelor) or with combined thromboxane receptor antagonists/synthetase inhibitors (e.g. terbogrel).

The dosage required to achieve such an effect is appropriately 3 to 30 mg/kg, preferably 1 to 10 mg/kg by intravenous route, and 5 to 50 mg/kg, preferably 3 to 30 mg/kg by oral route, in each case administered 1 to 4 times a day. For this purpose, the compounds of formula I prepared according to the invention may be formulated, optionally together with other active substances, with one or more inert conventional carriers and/or diluents, e.g. with corn starch, lactose, glucose, microcrystalline cellulose, magnesium stearate, polyvinylpyrrolidone, citric acid, tartaric acid, water, water/ethanol, water/glycerol, water/sorbitol, water/polyethylene glycol, propylene glycol, cetylstearyl alcohol, carboxymethylcellulose or fatty substances such as hard fat or suitable mixtures thereof, to produce conventional galenic preparations such as plain or coated tablets, capsules, powders, suspensions or suppositories.

The Examples which follow are intended to illustrate the invention:

### EXAMPLE 1

2-(5-carbamimidoyl-2-hydroxy-phenyl)-N-[3-methyl-4-(pyrrolidin-1-yl-carbonyl)-phenyl]-ethylamine-hydrochloride

a. 2-methyl-4-bromo-benzoic acid-pyrrolidinamide

35 g (0.163 mol) of 2-methyl-4-bromo-benzoic acid are dissolved in 1 l tetrahydrofuran and 100 ml water and combined with 57.8 g (0.18 mol) of O-(benzotriazol-1-yl)-N,N,N′,N′-tetramethyluronium tetrafluoroborate, 22.0 g (0.163 mol) of N-hydroxybenzotriazole and 62.7 ml (0.36 mol) of ethyl-dicyclohexylamine. After 10 minutes at ambient temperature 13.9 ml (0.167 mol) of pyrrolidine are added. The reaction mixture is stirred for 24 hours and evaporated down. The residue is combined with 5% saline solution/methylene chloride and extracted. The aqueous phase is extracted three times with methylene chloride, the combined organic phases are dried and evaporated down. The residue is purified on silica gel, eluting with methylene chloride plus ethanol (0-3%). The uniform fractions are combined and evaporated down.

Yield: 42 g (77% of theoretical),

R_{f} value: 0.45 (dichloromethane/ethanol=95:5)

b. N-[2-(5-bromo-2-methoxy-phenyl)-ethyl]-acetamide

1.9 g (9.8 mmol) of N-[2-(2-methoxy-phenyl)-ethyl]-acetamide are dissolved in 50 ml acetonitrile and after the addition of 2 g (11 mmol) of N-bromosuccinimide stirred for 4 hours at ambient temperature. Then the solvent is distilled off, the residue is stirred with dichloromethane and suction filtered. The mother liquor is evaporated down and chromatographed on silica gel, eluting with dichloromethane/methanol/ammonia (50:0.9:0.1).

Yield: 2.6 g (99% of theoretical),

R_{f} value: 0.47 (silica gel; dichloromethane/methanol/ammonia=24:0.9:0.1)

c. N-[2-(5-cyano-2-methoxy-phenyl)-ethyl]-acetamide

12.5 g (45.9 mmol) of N-[2-(5-bromo-2-methoxy-phenyl)-ethyl]-acetamide are dissolved in 50 ml dimethylformamide and after the addition of 8.2 g (91 mmol) of copper cyanide, 577 mg (0.5 mmol) of tetrakis-triphenylphosphine-palladium-(0) and 11.6 g aluminium oxide stirred for 20 hours under a nitrogen atmosphere at 140° C. The warm suspension is suction filtered and the mother liquor is evaporated down. The residue is chromatographed on silica gel, eluting with dichloromethane/ethanol (0-3%).

Yield: 4.9 g (49% of theoretical),

R_{f} value: 0.35 (silica gel; dichloromethane/methanol/ammonia=19:0.9:0.1)

d. (5-cyano-2-methoxy-phenyl)-ethylamine

4.9 g (22.4 mmol) of N-[2-(5-cyano-2-methoxy-phenyl)-ethyl]-acetamide are dissolved in 20 ml glacial acetic acid and after the addition of 60 ml of 3 molar hydrochloric acid refluxed for 15 hours. Then the solvent is distilled off, the residue is triturated in acetone and suction filtered. The crude product is dissolved in water, made alkaline with conc. ammonia and extracted with ethyl acetate. The organic phase is dried and evaporated down.

Yield: 2.6 g (66% of theoretical),

R_{f} value: 0.51 (silica gel; dichloromethane/methanol/ammonia=4:0.9:0.1)

e. 2-(5-cyano-2-methoxy-phenyl)-N-[3-methyl-4-(pyrrolidin-1-yl-carbonyl)-phenyl]-ethylamine

A solution of 2.0 g (6.7 mmol) of 2-methyl-4-bromo-benzoic acid-pyrrolidinamide and 1.9 g (8.5 mmol) of (5-cyano-2-methoxy-phenyl)-ethylamine in 75 ml toluene is combined under a nitrogen atmosphere with 5.7 g (17.5 mmol) of caesium carbonate, 120 mg (0.27 mmol) of palladium-I1-acetate and 240 mg (0.385 mmol) of 2,2′-bis-(diphenylphosphino)-1,1′-binaphthyl (BINAP) and heated to 130° C. for 18 hours. After cooling the reaction mixture is stirred with ice water and extracted with methylene chloride. The organic phase is washed with water, dried over magnesium sulphate and evaporated down. The crude product is purified on silica gel, eluting with methylene chloride/methanol/ammonia (1/0/0; 50/0.9/0.1 and 33/0.9/0.1).

Yield: 0.9 g (37% of theoretical),

R_{f} value: 0.71 (silica gel; dichloromethane/methanol/ammonia=9:0.9:0.1)

f. 2-(5-cyano-2-hydroxy-phenyl)-N-[3-methyl-4-(pyrrolidin-1-yl-carbonyl)-phenyl]ethylamine

0.5 g (1.3 mmol) of 2-(5-cyano-2-methoxy-phenyl)-N-[3-methyl-4-(pyrrolidin-1-yl-carbonyl)-phenyl]-ethylamine are dissolved in 40 ml dichloromethane and combined with 7 ml (7 mmol) of boron tribromide (1 M solution in dichloromethane) at −45 to −25° C. The reaction mixture is stirred for 20 hours at ambient temperature, combined with ice and conc. ammonia and extracted with dichloromethane/methanol (19:1). The combined organic extracts are evaporated down and chromatographed on silica gel, eluting with dichloromethane/ethanol (0-3%).

Yield: 0.2 g (46% of theoretical),

R_{f} value: 0.42 (silica gel; ethyl acetate/toluene/ammonia=9:0.9:0.1)

g. 2-(5-carbamimidoyl-2-hydroxy-phenyl)-N-[3-methyl-4-(pyrrolidin-1-yl-carbonyl)-phenyl]-ethylamine-hydrochloride

0.2 g (0.63 mmol) of 2-(5-cyano-2-hydroxy-phenyl)-N-[3-methyl-4-(pyrrolidin-1-yl-carbonyl)-phenyl]-ethylamine are dissolved in ethanolic hydrochloric acid and stirred for 4.75 hours at ambient temperature. The reaction mixture is evaporated down, taken up in 25 ml ethanol and combined with 0.9 g (9.5 mmol) of ammonium carbonate. After 18 hours at ambient temperature the undissolved material is filtered off and the filtrate evaporated down. The residue is triturated with ether, filtered, washed with ether and dried.

Yield: 0.2 g (87% of theoretical),

R_{f} value: 0.58 (Reversed Phase RP 8; 5% sodium chloride solution/methanol=1:2)

C₂₁H₂₆N₄O₂xHCl (366.47/402.93)

Mass spectrum: (M+H)⁺=367 (M+Cl)⁻=401/03 (Cl)

### EXAMPLE 2

N-(5-carbamimidoyl-2-hydroxy-benzyl)-3-methyl-4-(pyrrolidin-1-yl-carbonyl)-benzylamine-hydrochloride

a. 3-methyl-4-(pyrrolidin-1-yl-carbonyl)-benzonitrile

Prepared analogously to Example 1.c. from 2-methyl-4-bromo-benzoic acid-pyrrolidinamide, copper cyanide, tetrakis-triphenylphosphine-palladium-(0) and aluminium oxide in dimethylformamide.

Yield: 39% of theoretical,

R_{f} value: 0.22 (silica gel; cyclohexane/ethyl acetate=1:1)

b. 3-methyl-4-(pyrrolidin-1-yl-carbonyl)-benzylamine

2.3 g 3-methyl-4-(pyrrolidin-1-yl-carbonyl)-benzonitrile are dissolved in 75 ml ethanolic ammonia and after the addition of 0.4 g Raney nickel hydrogenated for 3 hours at 70° C. with hydrogen. Then the catalyst is filtered off and the filtrate is evaporated down.

Yield: 2.3 g (100% of theoretical),

R_{f} value: 0.45 (silica gel; dichloromethane/ethanol=9:1)

c. N-(5-cyano-2-hydroxy-benzyl)-3-methyl-4-(pyrrolidin-1-yl-carbonyl)-benzylamine

A solution of 1.1 g (5 mmol) of 3-methyl-4-(pyrrolidin-1-yl-carbonyl)-benzylamine in 10 ml methanol is combined with 0.3 ml (5 mmol) of glacial acetic acid and 0.2 g (3.5 mmol) of sodium cyanoborohydride. After 15 minutes 0.5 g (3.4 mmol) of 3-formyl-4-hydroxy-benzonitrile are added. The reaction mixture is stirred for 2 hours at ambient temperature and combined with ice and hydrochloric acid. By adding conc. ammonia the solution is adjusted to pH 8 and extracted with dichloromethane. The organic phase is evaporated down and chromatographed over silica gel, eluting with ethyl acetate.

Yield: 0.6 g (32% of theoretical),

R_{f} value: 0.33 (silica gel; dichloromethane/ethanol=19:1)

d. N-(5-carbamimidoyl-2-hydroxy-benzyl)-3-methyl-4-(pyrrolidin-1-yl-carbonyl)-benzylamine-hydrochloride

Prepared analogously to Example 1.g. from N-(5-cyano-2-hydroxy-benzyl)-3-methyl-4-(pyrrolidin-1-yl-carbonyl)-benzylamine and hydrochloric acid/ammonium carbonate in ethanol.

Yield: 98% of theoretical,

R_{f} value: 0.66 (Reversed Phase RP 8; 5% sodium chloride solution/methanol=1:1)

C₂₁H₂₆N₄O₂xHCl (366.47/402.93)

Mass spectrum: (M+H)⁺=367 (M−H)⁻=365 (M+Cl)⁻=401/03 (Cl)

The following compound is prepared analogously to Example 2:

(1) N-(5-carbamimidoyl-2-hydroxy-benzyl)-2,5-dimethyl-4-(pyrrolidin-1-yl-carbonyl)-benzylamine-dihydrochloride

Yield: 27% of theoretical,

R_{f} value: 0.6 (Reversed Phase RP 8; 5% sodium chloride solution/methanol 2:3)

C₂₂H₂₈N₄O₂x2HCl (380.49/453.41)

Mass spectrum: (M+H)⁺=381

### EXAMPLE 3

N-(5-carbamimidoyl-2-hydroxy-benzyl)-3-methyl-4-(pyrrolidin-1-yl-carbonyl)-benzamide-hydrochloride

a. 4-benzyloxy-3-hydroxymethyl-benzonitrile

A solution of 1.7 g (6.9 mmol) of 4-benzyloxy-3-formyl-benzonitrile in 10 ml tetrahydrofuran at 5-10° C. is added dropwise to a solution of 0.15 g (3.9 mmol) of sodium borohydride in 20 ml tetrahydrofuran. After 1.5 hours at 110° C. the solvent is distilled off. The residue is combined with 0.5 N sodium hydroxide solution and extracted with ethyl acetate. The organic phase is dried, evaporated down and crystallised with ether/petroleum ether.

Yield: 1.5 g (91% of theoretical),

R_{f} value: 0.2 (silica gel; petroleum ether/ethyl acetate=8:2)

b. 4-benzyloxy-3-(1,3-dioxo-1,3-dihydro-isoindol-2-yl)-methyl-benzonitrile

A solution of 2.6 g (15 mmol) of diethyl azodicarboxylate in 5 ml tetrahydrofuran is added dropwise at ambient temperature to a solution of 0.9 g (6.2 mmol) of phthalimide potassium salt, 1.5 g (6.2 mmol) of 4-benzyloxy-3-hydroxymethyl-benzonitrile and 3.9 g (15 mmol) of triphenylphosphine in 50 ml tetrahydrofuran, while the temperature rises to 42° C. After 24 hours the solvent is distilled off, the residue is taken up in sodium chloride solution/ethyl acetate and extracted with ethyl acetate. The combined organic extracts are dried and chromatographed on silica gel, eluting with petroleum ether/ethyl acetate (10:0, 9:1 and 8:2).

Yield: 0.7 g (31% of theoretical),

R_{f} value: 0.45 (silica gel; petroleum ether/ethyl acetate=7:3)

c. 4-benzyloxy-3-aminomethyl-benzonitrile

0.7 g (1.9 mmol) of 4-benzyloxy-3-(1,3-dioxo-1,3-dihydro-isoindol-2-yl)-methyl-benzonitrile are dissolved in 20 ml isopropanol and refluxed for 30 minutes with the addition of 1.5 ml of hydrazine hydrate. Then the reaction solution is evaporated down, the residue is stirred with ice water, suction filtered and dried.

Yield: 0.3 g (71% of theoretical),

R_{f} value: 0.1 (silica gel; petroleum ether/ethyl acetate=1:1)

d. 3-methyl-4-(pyrrolidin-1-carbonyl)-benzoic acid

26.8 g (0.1 mol) of 3-methyl-4-(pyrrolidin-1-carbonyl)-bromobenzene, 11.9 ml (0.13 mol) of n-butanol, 1 g (0.004 mol) of palladium-I1-acetate, 4.2 g (0.016 mol) of tri-phenylphosphine and 15.5 ml (0.12 mol) of N-ethyl-diisopropylamine are placed in a steel bomb and after the addition of carbon monoxide heated for 50 hours to 100° C. After cooling and evaporating off the carbon monoxide the reaction solution is stirred into ice water and extracted with ethyl acetate. The organic phase is dried and evaporated down. The residue is taken up in sodium hydrogen carbonate solution and ethyl acetate, the aqueous phase is adjusted to pH 4 with hydrochloric acid and extracted with ethyl acetate. The organic phases are dried and evaporated down.

Yield: 0.8 g (3.4% of theoretical),

R_{f} value: 0.4 (silica gel; dichloromethane/ethanol=19:1)

e. N-(2-benzyloxy-5-cyano-benzyl)-3-methyl-4-(pyrrolidin-1-yl-carbonyl)-benzamide-hydrochloride

Prepared analogously to Example 1.a. from 3-methyl-4-(pyrrolidin-1-carbonyl)-benzoic acid, O-(benzotriazol-1-yl)-N,N,N′,N′-tetramethyluronium tetrafluoroborate, N-methylmorpholine and 4-benzyloxy-3-aminomethyl-benzonitrile in dimethylformamide.

Yield: 93% of theoretical,

R_{f} value: 0.5 (silica gel; dichloromethane/ethanol=9:1)

f. N-(5-carbamimidoyl-2-benzyloxy-benzyl)-3-methyl-4-(pyrrolidin-1-yl-carbonyl)-benzamide-hydrochloride

Prepared analogously to Example 1.g. from N-(2-benzyloxy-5-cyano-benzyl)-3-methyl-4-(pyrrolidin-1-yl-carbonyl)-benzamide-hydrochloride and hydrochloric acid/ammonium carbonate in ethanol.

Yield: 0.3 g (77% of theoretical),

R_{f} value: 0.3 (silica gel; dichloromethane/ethanol/glacial acetic acid=8:2+1% glacial acetic acid)

g. N-(5-carbamimidoyl-2-hydroxy-benzyl)-3-methyl-4-(pyrrolidin-1-yl-carbonyl)-benzamide-hydrochloride

0.3 g (0.5 mmol) of N-(5-carbamimidoyl-2-benzyloxy-benzyl)-3-methyl-4-(pyrrolidin-1-yl-carbonyl)-benzamide-hydrochloride are dissolved in 50 ml methanol and after the addition of 200 mg palladium on activated charcoal (10%) hydrogenated with 5 atmospheres of hydrogen at ambient temperature. Then the catalyst is filtered off, the filtrate is evaporated down and triturated with petroleum ether/ether (1:1).

Yield: 120 mg (58% of theoretical),

C₂₁H₂₄N₄O₃xHCl (380.45/416.91)

Mass spectrum: (M+H)⁺=381 (M−H)⁻=379

The following compounds are prepared analogously to Example 3:

(1)N-(5-carbamimidoyl-2-hydroxy-benzyl)-2,5-dimethyl-4-(pyrrolidin-1-yl-carbonyl)-benzamide-hydrochloride

Yield: 81% of theoretical,

R_{f} value: 0.55 (Reversed Phase RP 8; 5% sodium chloride solution/methanol=2:3)

C₂₂H₂₆N₄O₃xHCl (394.48/430.94)

Mass spectrum: (M+H)⁺=395 (M−H)⁻=393 (M+Cl)⁻=429/31 (Cl)

(2) N-(3-carbamimidoyl-benzyl)-3-methyl-4-(pyrrolidin-1-yl-carbonyl)-benzamide-hydrochloride

Yield: 88% of theoretical,

R_{f} value: 0.53 (Reversed Phase RP 8; 5% sodium chloride solution/methanol 2:3)

C₂₁H₂₅N₄O₂xHCl (364.45/400.91)

Mass spectrum: (M+H)⁺=365 (M+Cl)⁻=399/01 (Cl)

(3) N-(5-carbamimidoyl-2-hydroxy-benzyl)-3-trifluoromethyl-4-(pyrrolidin-1-yl-carbonyl)-benzamide-hydrochloride

Yield: 87% of theoretical,

R_{f} value: 0.7 (Reversed Phase RP 8; 5% sodium chloride solution/methanol 2:8)

C₂₁H₂₁F₃N₄O₃xHCl (434.42/470.88)

Mass spectrum: (M+H)⁺=435 (M−H)⁻=433

### EXAMPLE 4

N-(5-aminomethyl-2-hydroxy-benzyl)-3-methyl-4-(pyrrolidin-1-yl-carbonyl)-benzamide

Prepared analogously to Example 2.b. from N-(5-cyano-2-benzyloxy-benzyl)-3-methyl-4-(pyrrolidin-1-yl-carbonyl)-benzamide in methanolic ammonia/Raney nickel/hydrogen and subsequent reaction analogously to Example 3.g. with hydrogen in methanol with the addition of palladium on activated charcoal.

Yield: 34% of theoretical,

R_{f} value: 0.35 (silica gel; dichloromethane/ethanol=8:2)

C₂₁H₂₅N₃O₃ (367.45)

Mass spectrum: (M−H)⁻=366

The following compounds are prepared analogously to Example 4:

(1)2-(3-aminomethyl-phenyl)-2-[3-methyl-4-(pyrrolidin-1-yl-carbonyl)-phenyl]-acetic acid-N-ethylamide-hydrochloride

Yield: 91% of theoretical,

R_{f} value: 0.13 (silica gel; ethyl acetate/ethanol=3:2+1% ammonia)

C₂₄H₃₀N₄O₃xHCl (422.53/458.99)

Mass spectrum: (M+H)⁺=423

(2) 3-(3-aminomethyl-phenyl)-3-[3-methyl-4-(pyrrolidin-1-yl-carbonyl)-benzoylamino]-propionic acid-N-ethylamide

Yield: 28% of theoretical,

R_{f} value: 0.22 (silica gel; dichloromethane/ethanol=9:1+1% ammonia)

C₂₅H₃₂N₄O₃ (436.56)

Mass spectrum: (M+H)⁺=437 (M−H)⁻=435

### EXAMPLE 5

N-(5-carbamimidoyl-2-hydroxy-benzyl)-3-methyl-4-[N-cyclopentyl-N-(3-ethoxycarbonyl-propionyl)amino]-benzamide-hydrochloride

a. benzyl 4-cyclopentylamino-3-methyl-benzoate

3.3 g (13.6 mmol) of benzyl 4-amino-3-methyl-benzoate, 1.3 ml (15 mmol) of cyclo-pentanone, 1.2 ml (20.5 mmol) of glacial acetic acid and 0.1 g of p-toluenesulphonic acid are dissolved in 70 ml tetrahydrofuran and stirred for 30 minutes at ambient temperature. Then 4.0 g (17.8 mmol) of sodium triacetoxyborohydride are added. After 26 hours at ambient temperature the solvent is distilled off and the residue is distributed in water/ethyl acetate. The aqueous phase is extracted three times with ethyl acetate. The combined organic extracts are dried and purified over silica gel, eluting with dichloromethane.

Yield: 0.8 g (19% of theoretical),

R_{f} value: 0.78 (silica gel; dichloromethane/ethanol=95:5)

b. benzyl 4-[cyclopentyl-(3-ethoxycarbonyl-propionyl)-amino]-3-methyl-benzoate

A solution of 0.8 g (2.6 mmol) of benzyl 4-cyclopentylamino-3-methyl-benzoate in 30 ml tetrahydrofuran is combined with 0.1 g (2.6 mmol) of sodium hydride and heated to 40° C. for one hour. After the addition of 0.3 ml (2.34 mmol) of ethyl succinate chloride the reaction mixture is stirred for 5 days at ambient temperature. After evaporation of the solvent the residue is taken up in ethyl acetate, washed with saline solution and dried. The crude product is purified on silica gel, eluting with dichloromethane.

Yield: 0.8 g (73% of theoretical),

R_{f} value: 0.64 (silica gel; dichloromethane/ethanol=95:5)

c. 4-[cyclopentyl-(3-ethoxycarbonyl-propionyl)-amino]-3-methyl-benzoic acid

Prepared analogously to Example 3.g. from benzyl 4-[cyclopentyl-(3-ethoxycarbonyl-propionyl)-amino]-3-methyl-benzoate and palladium on activated charcoal/hydrogen in methanol.

Yield: 91% of theoretical,

R_{f} value: 0.12 (silica gel; dichloromethane/ethanol 95:5)

d. N-(5-cyano-2-benzyloxy-benzyl)-3-methyl-4-[N-cyclopentyl-(3-ethoxycarbonyl-propionyl)amino]-benzamide

Prepared analogously to Example 1.a. from 4-[cyclopentyl-(3-ethoxycarbonyl-propionyl)-amino]-3-methyl-benzoic acid, 4-benzyloxy-3-aminomethyl-benzonitrile, O-(benzotriazol-1-yl)-N,N,N′,N′-tetramethyluronium tetrafluoroborate and N-methylmorpholine in dimethylformamide.

Yield: 95% of theoretical,

R_{f} value: 0.28 (silica gel; dichloromethane/ethanol=95:5)

e. N-(5-carbamimidoyl-2-hydroxy-benzyl)-3-methyl-4-[N-cyclopentyl-(3-ethoxycarbonyl-propionyl)amino]-benzamide-hydrochloride

Prepared analogously to Example 1.g. from N-(5-cyano-2-benzyloxy-benzyl)-3-methyl-4-[N-cyclopentyl-(3-ethoxycarbonylpropionyl)amino]-benzamide and hydrochloric acid/ammonium carbonate in ethanol and subsequent reaction analogously to Example 3.g. with hydrogen in methanol with the addition of palladium on activated charcoal.

Yield: 51% of theoretical,

R_{f} value: 0.31 (Reversed Phase RP 8; 5% sodium chloride solution/methanol 6:4)

C₂₇H₃₄N₄O₅xHCl (494.60/531.06)

Mass spectrum: (M+H)⁺=495 (M+Cl)⁻=529/31 (Cl)

The following compounds are prepared analogously to Example 5:

(1) N-(5-carbamimidoyl-2-hydroxy-benzyl)-3-methyl-4-(N-acetyl-N-cyclobutyl-amino-benzamide-hydrochloride

Yield: 97% of theoretical,

R_{f} value: 0.12 (silica gel; dichloromethane/ethanol=4:1)

C₂₂H₂₆N₄O₃xHCl (394.48/430.94)

Mass spectrum: (M+H)⁺=395 (M−H)⁻=393 (M+Cl)⁻=429/31 (Cl)

(2) N-(5-carbamimidoyl-2-hydroxy-benzyl)-3-methyl-4-(N-cyclopentyl-N-methylamino)-benzamide-hydrochloride

Yield: 91% of theoretical,

R_{f} value: 0.30 (Reversed Phase RP 8; 5% sodium chloride solution/methanol=2:3)

C₂₂H₂₈N₄O₂xHCl (380.49/416.95)

Mass spectrum: (M+H)⁺=381 (M−H)⁻=379 (M+Cl)⁻=415/17 (Cl)

### EXAMPLE 6

N-(5-carbamimidoyl-2-hydroxy-benzyl)-3-methyl-4-[N-cyclopentyl-N-(3-carboxy-propionyl)amino]-benzamide-hydrochloride

0.2 g (0.28 mmol) of N-(5-carbamimidoyl-2-hydroxy-benzyl)-3-methyl-4-[N-cyclopentyl-(3-ethoxycarbonylpropionyl)amino]-benzamide-hydrochloride are stirred in 5 ml of 6 molar hydrochloric acid at ambient temperature for 4 hours. The solvent is distilled off and the residue is purified on Reversed Phase RP 8, eluting with water/methanol (0-50%).

Yield: 99% of theoretical,

R_{f} value: 0.49 (Reversed Phase RP 18; 5% sodium chloride solution/methanol=6:4)

C₂₅H₃₀N₄O₅xHCl (466.54/503.00)

Mass spectrum: (M+H)⁺=467 (M−H)⁻=465 (M+Na)⁺=489

### EXAMPLE 7

N-(5-carbamimidoyl-2-hydroxy-benzyl)-4-cyclopentylamino-3-methyl-benzamide-hydrochloride

a. methyl 4-cyclopentylamino-3-methyl-benzoate

Prepared analogously to Example 1.e. from methyl 4-bromo-3-methyl-benzoate, cyclopentylamine, caesium carbonate, palladium-II-acetate and 2,2′-bis-(diphenyl-phosphino)-1,1 ′-binaphthyl in toluene.

Yield: 95% of theoretical,

R_{f} value: 0.55 (silica gel; dichloromethane)

b. 4-cyclopentylamino-3-methyl-benzoic acid

3.3 g (14 mmol) of methyl 4-cyclopentylamino-3-methyl-benzoate are dissolved in 5 ml methanol and combined with 30 ml of sodium hydroxide solution (2N). After 12 hours at ambient temperature the reaction mixture is evaporated down and combined with 30 ml hydrochloric acid (2N) with cooling. After 30 minutes the solution is combined with dichloromethane and extracted. The organic phase is dried and evaporated down.

Yield: 0.8 g (26% of theoretical),

R_{f} value: 0.74 (silica gel; petroleum ether/ethyl acetate=4:6)

c. N-(2-benzyloxy-5-cyano-benzyl)-4-cyclopentylamino-3-methyl-benzamide

Prepared analogously to Example 1.a. from 4-cyclopentylamino-3-methyl-benzoic acid, O-(benzotriazol -1-yl)-N,N,N′-N′-tetramethyluronium fluoroborate, N-methylmorpholine and 4-benzyloxy-3-aminomethyl-benzonitrile in dimethylformamide.

Yield: 49% of theoretical,

R_{f} value: 0.77 (silica gel; dichloromethane/ethanol 95:5)

d. N-(5-carbamimidoyl-2-hydroxy-benzyl)-4-cyclopentylamino-3-methyl-benzamide-hydrochloride

Prepared analogously to Example 1.g. from N-(2-benzyloxy-5-cyano-benzyl)-4-cyclopentylamino-3-methyl-benzamide and hydrochloric acid/ammonium carbonate in ethanol and subsequent reaction with hydrogen/palladium on activated charcoal in methanol analogously to Example 3.g.

Yield: 78% of theoretical,

R_{f} value: 0.29 (silica gel; dichloromethane/ethanol 4:1)

C₂₁H₂₆N₄O₂xHCl (366.47/402.93)

Mass spectrum: (M+H)⁺=367 (M−H)⁻=365 (M+Cl)⁻=401/03 (Cl)

### EXAMPLE 8

Ethyl 2-(3-carbamimidoyl-phenyl)-2-[3-methyl-4-(pyrrolidin-1-yl-carbonyl)-benzoylamino]-acetate

a. benzyl tert-butoxycarbonylamino-(3-cyano-phenyl)-acetate

Prepared analogously to Example 1.c. from benzyl tert-butoxycarbonylamino-(3-bromo-phenyl)-acetate and copper-(I)-cyanide/tetrakis-triphenylphosphine-palladium-(0).

Yield: 41% of theoretical,

R_{f} value: 0.25 (silica gel; cyclohexane/ethyl acetate=4:1)

b. benzyl amino-(3-cyano-phenyl)-acetate

Prepared analogously to Example 1.d. from benzyl tert-butoxycarbonylamino-(3-cyano-phenyl)-acetate and hydrochloric acid in dioxan.

Yield: 66% of theoretical,

R_{f} value: 0.4 (silica gel; dichloromethane/methanol 95:5+ammonia)

c. benzyl (3-cyano-phenyl)-{[3-methyl-4-(pyrrolidin-1-yl-carbonyl)-phenyl]-carbonylamino}-acetate

Prepared analogously to Example 1.a. from benzyl amino-(3-cyano-phenyl)-acetate and 3-methyl-4-(pyrrolidin-1-carbonyl)-benzoic acid, O-(benzotriazol-1-yl)-N,N,N′,N′-tetramethyluronium tetrafluoroborate and N-methylmorpholine in dimethylformamide.

Yield: 93% of theoretical,

R_{f} value: 0.5 (silica gel; ethyl acetate)

d. Ethyl (3-carbamimidoyl-phenyl)-{[3-methyl-4-(pyrrolidin-1-yl-carbonyl)-phenyl]-carbonyl-aminol}-acetate

Prepared analogously to Example 1.g. from benzyl (3-cyano-phenyl)-{[3-methyl-4-(pyrrolidin-1-yl-carbonyl)-phenyl]-carbonyl-amino }-acetate and hydrochloric acid/ammonium carbonate in ethanol.

Yield: 47% of theoretical,

R_{f} value: 0.46 (Reversed Phase RP8; 5% saline solution/methanol=2:3)

C₂₄H₂₈N₄O₄xCH₃COOH (436.52/496.57)

Mass spectrum: (M+H)⁺=437 (M−H)⁻=435

### EXAMPLE 9

2-(3-carbamimidoyl-phenyl)-2-[3-methyl-4-(pyrrolidin-1-yl-carbonyl)-benzoyl-amino]-acetic acid-hydrochloride

Prepared analogously to Example 7.b. from ethyl (3-carbamimidoyl-phenyl)-{[3-methyl-4-(pyrrolidin-1-yl-carbonyl)-phenyl]-carbonyl-amino }-acetate and sodium hydroxide solution.

Yield: 91% of theoretical,

R_{f} value: 0.55 (Reversed Phase RP8; 5% saline solution/methanol 2:3)

C₂₂H₂₄N₄O₄xHCl (408.46/444.92)

Mass spectrum: (M+H)⁺=409 (M+Na)⁺=431

### EXAMPLE 10

N-(5-carbamimidoyl-2-hydroxy-benzyl)-3-methyl-4-[N-(2-aminoacetyl)-N-cyclopentyl-amino]-benzamide-hydrochloride

a. methyl 4-{cyclopentyl-[2-(2,2,2-trifluoro-acetylamino)-acetyl]-amino}-3-methyl-benzoate

Prepared analogously to Example 5.b. from methyl 4-cyclopentylamino-3-methyl-benzoate, (2,2,2-trifluoro-acetylamino)-acetyl chloride and sodium hydride in tetrahydrofuran.

Yield: 46% of theoretical,

R_{f} value: 0.65 (silica gel; dichloromethane/ethanol=95:5)

b. 4-[N-(2-benzyloxycarbonylamino-acetyl)-cyclopentyl-amino]-3-methyl-benzoic acid

1.5 g (3.8 mmol) of methyl 4-{cyclopentyl-[2-(2,2,2-trifluoro-acetyl-amino)-acetyl]-amino}-3-methyl-benzoate are stirred in 20 ml methanol and 7.8 ml (7.7 mmol) of 1 molar sodium hydroxide solution for 2 hours. The solvent is distilled off, the residue is combined with 3.9 ml (3.8 mmol) of 1 molar sodium hydroxide solution. Then 0.6 ml (4.1 mmol) of benzyl chloroformate are added dropwise. After 1.5 hours the mixture is acidified with 1 molar hydrochloric acid and extracted with ethyl acetate. The combined organic extracts are dried and evaporated down.

Yield: 1.3 g (80% of theoretical),

R_{f} value: 0.10 (silica gel; dichloromethane/ethanol=95:5)

c. N-(2-benzyloxy-5-cyano-benzyl)-3-methyl-4-[N-(2-benzyloxycarbonylamino-acetyl)-cyclopentyl-amino]-benzamide

Prepared analogously to Example 1.a. from 4-[N-(2-benzyloxycarbonyl-amino-acetyl)-cyclopentyl-amino]-3-methyl-benzoic acid, 3-aminomethyl-4-benzyloxy-benzonitrile, O-(benzotriazol-1-yl)-N,N,N′,N′-tetramethyl-uronium tetrafluoroborate and N-methylmor-pholine in dimethylformamide.

Yield: 66% of theoretical,

R_{f} value: 0.68 (silica gel; dichloromethane/ethanol 95:5)

d. N-(5-carbamimidoyl-2-hydroxy-benzyl)-3-methyl-4-[N-(2-aminoacetyl)-cyclopentyl-amino)]-benzamide-hydrochloride

Prepared analogously to Example 1.g. from N-(2-benzyloxy-5-cyano-benzyl)-3-methyl-4-[N-(2-benzyloxycarbonylamino-acetyl)-cyclopentyl-amino]-benzamide and hydrochloric acid/ammonium carbonate in ethanol followed by reaction with hydrogen/palladium on activated charcoal in methanol analogously to Example 3.g.

Yield: 55% of theoretical,

R_{f} value: 0.61 (Reversed Phase RP 8; 5% sodium chloride solution/methanol=2:3)

C₂₃H₂₉N₅O₃xHCl (423.52/459.98)

Mass spectrum: (M+H)⁺=424 (M+Cl)⁻=458/60 (Cl)

The following compound is prepared analogously to Example 10:

(1) N-(5-carbamimidoyl-2-hydroxy-benzyl)-3-methyl-4-[N-(3-amino-propionyl)-N-cylopentyl-amino]-benzamide-hydrochloride

Yield: 100% of theoretical,

R_{f} value: 0.53 (Reversed Phase RP 8; 5% sodium chloride solution/methanol=2:3)

C₂₄H₃₁N₅O₃xHCl (437.55/474.01)

Mass spectrum: (M+H)⁺=438 (M+Cl)⁻=472/74 (Cl)

### EXAMPLE 11

N-(5-carbamimidoyl-2-hydroxy-benzyl)-3-chloro-4-(pyrrolidin-1-yl-carbonyl)-benzamide-hydrochloride

a. 2-Chloro-4-(pyrrolidin-1-yl-carbonyl)-benzoic acid

0.9 g (4.3 mmol) of 2-chloro-terephthalic acid and 0.8 g (4.7 mmol) of N,N′-carbonyldiimidazole are stirred in 10 ml dimethylformamide for 15 minutes. Then 0.5 ml (6.5 mmol) of pyrrolidine and 1.0 ml (9.5 mmol) of N-methylmorpholine are added. After 48 hours at ambient temperature the solvent is distilled off and the residue is chromatographed on silica gel, eluting with dichloromethane/ethanol/glacial acetic acid 95:5:0.02 and 80:20:0.02.

Yield: 0.3 g (23% of theoretical),

R_{f} value: 0.21 (silica gel; dichloromethane/ethanol 95:5+glacial acetic acid)

b. N-(2-benzyloxy-5-cyano-benzyl)-3-chloro-4-(pyrrolidin-1-yl-carbonyl)-benzamide

Prepared analogously to Example 1.a. from 2-chloro-4-(pyrrolidin-1-yl-carbonyl)-benzoic acid, 3-aminomethyl-4-benzyloxy-benzonitrile, O-(benzotriazol-1-yl)-N,N,N′,N′-tetramethyluronium tetrafluoroborate and N-ethyldiisopropylamine in tetrahydro furan.

Yield: 73% of theoretical,

R_{f} value: 0.45 (silica gel; dichloromethane/ethanol=95:5)

c. N-(5-carbamimidoyl-2-hydroxy-benzyl)-3-chloro-4-(pyrrolidin-1-yl-carbonyl)-benzamide-hydrochloride

Prepared analogously to Example 1.g. from N-(2-benzyloxy-5-cyano-benzyl)-3-chloro-4-(pyrrolidin-1-yl-carbonyl)-benzamide and hydrochloric acid/ammonium carbonate in ethanol followed by reaction with hydrogen/palladium on activated charcoal in methanol analogously to Example 3.g.

Yield: 66% of theoretical,

R_{f} value: 0.54 (Reversed Phase RP 8; 5% sodium chloride solution/methanol 2:3)

C₂₀H₂₁CIN₄O₃xHCl (400.87/437.34)

Mass spectrum: (M+H)⁺=401 (M−H)⁻=399 (M+Cl)⁻=435/7/9 (Cl₂)

### EXAMPLE 12

Ethyl 3-(3-carbamimidoyl-phenyl)-3-[3-methyl-4-(pyrrolidin-1-yl-carbonyl)-benzoylamino]-propionate-hydrochloride

a. 3-amino-3-(3-cyano-phenyl)-propionic acid

13.1 g (0.1 mol) of 3-cyanobenzaldehyde are dissolved in 50 ml of 95% ethanol and after the addition of 15.4 g (0.2 mol) of ammonium acetate stirred for 15 minutes at 45° C. Then 20.8 g (0.2 mol) of malonic acid in 50 ml of 95% ethanol are added dropwise. The reaction mixture is refluxed for 2 hours. The crystalline product is suction filtered and recrystallised from methanol/water.

Yield: 6.5 g (34% of theoretical),

C₁₀H₁₀N₂O₂ (190.20)

Mass spectrum: (M+H)⁺=191 (M−H)⁻=189

b. 3-(3-cyano-phenyl)-3-[3-methyl-4-(pyrrolidin-1-yl-carbonyl)-benzoylamino]-propionic acid

380.4 mg (2 mmol) of 3-amino-3-(3-cyano-phenyl)-propionic acid are added to 2.0 ml of 2 molar sodium hydroxide solution while cooling with ice. After the addition of 500 mg (1.98 mmol) of 3-methyl-4-(pyrrolidin-1-yl-carbonyl)-benzoylchloride the reaction mixture is stirred for 4 hours at ambient temperature. The solution is diluted with water and adjusted to pH 4 with 1 M hydrochloric acid. The precipitate formed is suction filtered and chromatographed on silica gel, eluting with dichloromethane/ethanol (4-10%).

Yield: 280 mg (35% of theoretical),

R_{f} value: 0.35 (silica gel; dichloromethane/ethanol=9:1)

c. Ethyl 3-(3-carbamimidoyl-phenyl)-3-[3-methyl-4-(pyrrolidin-1-yl-carbonyl)-benzoyl-amino]-propionate hydrochloride

Prepared analogously to Example 1.g. from 3-(3-cyano-phenyl)-3-[3-methyl-4-(pyrrolidin-1-yl-carbonyl)-benzoylamino]-propionic acid and hydrochloric acid/ammonium carbonate in ethanol.

Yield: 59% of theoretical,

R_{f} value: 0.40 (Reversed Phase RP 8; 5% sodium chloride solution/methanol=2:3)

C₂₅H₃₀N₄O₄xHCl (450.54/487.01)

Mass spectrum: (M+H)⁺=451 (M−H)⁻=449 (M+Cl)⁻=585/87 (Cl)

The following compounds are prepared analogously to Example 12:

(1) ethyl 3-(3-carbamimidoyl-phenyl)-3-[3-chloro-4-(pyrrolidin-1-yl-carbonyl)-benzoylamino]-propionate-hydrochloride

Yield: 98% of theoretical,

R_{f} value: 0.22 (silica gel; dichloromethane/ethanol=4:1)

C₂₄H₂₇CIN₄O₄xHCl (470.69/507.43)

Mass spectrum: (M+H)⁺=471/73 (Cl) (M+Cl)⁻=505/7/9 (Cl₂)

(2) ethyl 3-(3-carbamimidoyl-phenyl)-3-{3-methyl-4-[N-(3-amino-propionyl)-N-cyclopentyl-amino]-benzoylamino}-propionate-hydrochloride

Yield: 100% of theoretical,

R_{f} value: 0.28 (Reversed Phase RP 8; 5% sodium chloride solution/methanol=2:3)

C₂₈H₃₇N₅O₄xHCl (507.63/544.11)

Mass spectrum: (M+H)⁺=508 (M+Cl)⁻=542/44 (Cl)

(3) ethyl 3-(3-carbamimidoyl-phenyl)-3-[3-bromo-4-(pyrrolidin-1-yl-carbonyl-benzoylamino]-propionate-hydrochloride

Yield: 81% of theoretical,

R_{f} value: 0.70 (Reversed Phase RP 8; 5% sodium chloride solution/methanol 1:4)

C₂₄H₂₇BrN₄O₄xHCl (515.41/551.87)

Mass spectrum: (M+H)⁺=515/17 (Br)

(4) ethyl 3-(3-carbamimidoyl-phenyl)-3-[3-methyl-4-(2,5-dihydropyrrol-1-yl-carbonyl)-benzoylamino]-propionate-hydrochloride

Yield: 45% of theoretical,

R_{f} value: 0.30 (silica gel; dichloromethane/ethanol=4:1+1% glacial acetic acid)

C₂₅H₂₈N₄O₄xHCl (448.51/484.91)

Mass spectrum: (M+H)⁺=449 (M+Cl⁻)⁻=483/5 (Cl)

(5) ethyl 3-(3-carbamimidoyl-phenyl)-3-[3-ethynyl-4-(pyrrolidin-1-yl-carbonyl)-benzoyl-amino]-propionate-hydrochloride

Yield: 71% of theoretical,

R_{f} value: 0.20 (silica gel; dichloromethane/ethanol=3:1)

C₂₆H₂₈N₄O₄xHCl (460.53/497.01)

Mass spectrum: (M+H)⁺=461 (M+Cl⁻)⁻=495/7 (Cl)

(6) ethyl 3-(3-carbamimidoyl-phenyl)-3-[3-ethyl-4-(pyrrolidin-1-yl-carbonyl)-benzoylamino]-propionate-hydrochloride

Yield: 38% of theoretical,

R_{f} value: 0.18 (silica gel; dichloromethane/ethanol=4:1)

C₂₆H₃₂N₄O₄xHCl (464.56/501.03)

Mass spectrum: (M+H)⁺=465 (M+Cl⁻)⁻=499/501 (Cl)

(7) ethyl 3-(3-carbamimidoyl-phenyl)-3-[3-ethenyl-4-(pyrrolidin-1-yl-carbonyl)-benzoyl-amino]-propionate-hydrochloride

Yield:

R_{f} value:

C₂₆H₃₀N₄O₄xHCl (462.55/499.0)

Mass spectrum:

### EXAMPLE 13

3-(3-carbamimidoyl-phenyl)-3-[3-methyl-4-(pyrrolidin-1-yl-carbonyl)-benzoylamino]-propionic acid-hydrochloride

Prepared analogously to Example 6 from ethyl 3-(3-carbamimidoyl-phenyl)-3-[3-methyl-4-(pyrrolidin-1-yl-carbonyl)-benzoylamino]-propionate-hydrochloride and 6 molar hydrochloric acid.

Yield: 85% of theoretical,

R_{f} value: 0.50 (Reversed Phase RP 8; 5% sodium chloride solution/methanol=2:3)

C₂₃H₂₆N₄O₄xHCl (422.49/458.96)

Mass spectrum: (M+H)⁺=423 (M−H)⁻=421

The following compounds are obtained analogously to Example 13:

(1) 3-(3-carbamimidoyl-phenyl)-3-[3-trifluoromethyl-4-(pyrrolidin-1-yl-carbonyl)-benzoylamino]-propionic acid-hydrochloride

Yield: 65% of theoretical,

R_{f} value: 0.5 (Reversed Phase RP 8; 5% sodium chloride solution/methanol=2:3)

C₂₃H₂₃F₃N₄O₄xHCl (476.46/512,91)

Mass spectrum: (M+H)⁺=477 (M−H)⁻=475

(2) 3-(3-carbamimidoyl-phenyl)-3-[3-chloro-4-(pyrrolidin-1-yl-carbonyl)-benzoylamino]-propionic acid-hydrochloride

Yield: 90% of theoretical,

R_{f} value: 0.42 (Reversed Phase RP 8; 5% sodium chloride solution/methanol=2:3)

C₂₂H₂₃CIN₄O₄xHCl (442.9/479.38)

Mass spectrum: (M+H)⁺=443/5 (Cl) (M−H)⁻=441/3 (Cl)

(3) 3-(3-carbamimidoyl-phenyl)-3-[3-methyl-4-(2.5-dihydropyrrol-1-yl-carbonyl)-benzoylamino]-propionic acid-hydrochloride

Yield: 27% of theoretical,

R_{f} value: 0.40 (Reversed Phase RP 8; 5% sodium chloride solution/methanol=2:3)

C₂₃H₂₄N₄O₄xHCl (420.46/456.93)

Mass spectrum: (M+H)⁺=421 (M−H)⁻=419

(4) 3-(3-carbamimidoyl-phenyl)-3-[3-ethynyl-4-(pyrrolidin-1-yl-carbonyl)-benzoylamino]-propionic acid-hydrochloride

Yield: 86% of theoretical,

R_{f} value: 0.15 (Reversed Phase RP 8; 5% sodium chloride solution/methanol=2:3)

C₂₄H₂₄N₄O₄xHCl (432.88/468.95)

Mass spectrum: (M+H)⁺=433 (M−H)⁻=431 (M+Cl)⁻=467/9

(5) 3-(3-carbamimidoyl-phenyl)-3-[3-ethyl-4-(pyrrolidin-1-yl-carbonyl)-benzoylamino]-propionic acid-hydrochloride

Yield: 85% of theoretical,

R_{f} value: 0.57 (Reversed Phase RP 8; 5% sodium chloride solution/methanol 2:3)

C₂₄H₂₈N₄O₄xHCl (436.51/472.98)

Mass spectrum: (M+H)⁺=437 (M−H)⁻=435 (M+Cl)⁻=471/3

(6) 3-[3-methyl-4-(pyrrolidin-1-yl-carbonyl)-benzoylamino]-3-(3-carbamimidoyl-phenyl)-propionic acid-N-methyl-N-(hydroxycarbonylmethyl)-amide-hydrochloride

Yield: 42% of theoretical,

R_{f} value: 0.35 (Reversed Phase RP 8; 5% sodium chloride solution/methanol=2:3)

C₂₆H₃₁N₅O₅xHCl (493.56/530.03)

Mass spectrum: (M+H)⁺=494 (M−H)⁻=492 (M+Cl)⁻=528/30

(7) 3-[3-methyl-4-(pyrrolidin-1-yl-carbonyl)-benzoylamino]-3-(3-carbamimidoyl-phenyl)-propionic acid-N-(hydroxycarbonylmethyl)-N-(n-propyl)-amide-hydrochloride

Yield: 67% of theoretical,

R_{f} value: 0.33 (Reversed Phase RP 8; 5% sodium chloride solution/methanol=2:3)

C₂₈H₃₅N₅O₅xHCl (521.62/558.08)

Mass spectrum: (M+H)⁺=522 (M−H)⁻=520

(8) 3-(3-carbamimidoyl-phenyl)-3-[3-ethenyl-4-(pyrrolidin-1-yl-carbonyl)-benzoyl-amino]-propionic acid-hydrochloride

Yield:

R_{f} value:

C₂₄H₂₆N₄O₄xHCl (434.50/470.95)

Mass spectrum:

### EXAMPLE 14

3-(3-carbamimidoyl-phenyl)-3-[3-methyl-4-(pyrrolidin-1-yl-carbonyl)-benzoylamino]-propionic acid-N,N-dimethylamide-hydrochloride

a. 3-(3-cyano-phenyl)-3-[3-methyl-4-(pyrrolidin-1-yl-carbonyl)-benzoylamino]-propionic acid dimethylamide

Prepared analogously to Example 1.a. from 3-(3-cyano-phenyl)-3-[3-methyl-4-(pyrrolidin-1-yl-carbonyl)-benzoylamino]-propionic acid, dimethylamine, O-(benzotriazol-1-yl)-N,N,N′,N′-tetramethyl-uronium tetrafluoroborate and N-methylmorpholine in dimethylformamide.

Yield: 36% of theoretical,

R_{f} value: 0.38 (silica gel; dichloromethane/ethanol=19:1)

b. 3-(3-carbamimidoyl-phenyl)-3-[3-methyl-4-(pyrrolidin-1-yl-carbonyl)-benzoylamino]-propionic acid dimethylamide-hydrochloride

Prepared analogously to Example 1.g. from 3-(3-cyano-phenyl)-3-[3-methyl-4-(pyrrolidin-1-yl-carbonyl)-benzoylamino]-propionic acid-dimethylamide and hydrochloric acid/ammonium carbonate in ethanol.

Yield: 24% of theoretical,

R_{f} value: 0.17 (silica gel; dichloromethane/ethanol 4:1+1% glacial acetic acid)

C₂₅H₃₁N₅O₃xHCl (449.56/486.03)

Mass spectrum: (M+H)⁺=450 (M+Cl)⁻=484/86 (Cl)

The following are prepared analogously to Example 14:

(1) N-[1-(3-carbamimidoyl-phenyl)-2-oxo-2-(pyrrolidin-1-yl)-ethyl]-3-methyl-4-(pyrrolidin-1-yl-carbonyl)-benzamide-hydrochloride

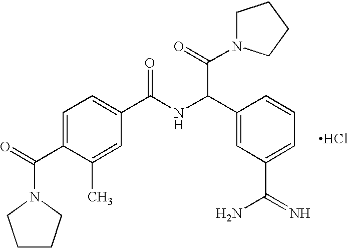

Yield: 79% of theoretical

R_{f} value: 0.40 (Reversed Phase RP 8; 5% sodium chloride solution/methanol=2:3)

C₂₆H₃₁N₅O₃xHCl (461.53/498.03)

Mass spectrum: (M+H)⁺=462

(2) 2-(3-carbamimidoyl-phenyl)-2-[3-methyl-4-(pyrrolidin-1-yl-carbonyl)-benzoylamino]-acetic acid-N,N-dimethylamide-hydrochloride

Yield: 76% of theoretical

R_{f} value: 0.46 (Reversed Phase RP 8; 5% sodium chloride solution/methanol=2:3)

C₂₄H₂₉N₅O₃xHCl (435.53/471.99)

Mass spectrum: (M+H)⁺=436

(3) 2-(3-carbamimidoyl-phenyl)-2-[3-methyl-4-(pyrrolidin-1-yl-carbonyl)-benzoylamino]-acetic acid-N-ethylamide-hydrochloride

Yield: 56% of theoretical

R_{f} value: 0.38 (Reversed Phase RP 8; 5% sodium chloride solution/methanol=2:3)

C₂₄H₂₉N₅O₃xHCl (435.53/471.99)

Mass spectrum: (M+H)⁺=436

(4) 3-(3-carbamimidoyl-phenyl)-3-[3-methyl-4-(pyrrolidin-1-yl-carbonyl)-benzoylamino]-propionic acid-N-ethylamide-hydrochloride

Yield: 43% of theoretical

R_{f} value: 0.25 (silica gel; dichloromethane/ethanol=4:1+1% glacial acetic acid)

C₂₅H₃₁N₅O₃xHCl (449.55/486.02)

Mass spectrum: (M+H)⁺=450 (M−H)⁻=448 (M+Cl)⁻=484/6 (Cl)

(5) 3-[3-methyl-4-(pyrrolidin-1-yl-carbonyl)-benzoylamino]-3-(3-carbamimidoyl-phenyl)-propionic acid-N-(ethoxycarbonylmethyl)-N-(n-propyl)-amide-hydrochloride

Yield: 91% of theoretical

R_{f} value: 0.27 (silica gel; dichloromethane/ethanol=4:1+1% glacial acetic acid)

C₃₀H₃₉N₅O₃xHCl (549.67/586.14)

Mass spectrum: (M+H)⁺=550 (M+Cl)⁻=584/6 (Cl)

### EXAMPLE 15

N-[1-(5-carbamimidoyl-2-hydroxy-phenyl)-ethyl]-3-methyl-4-(pyrrolidin-1-yl-carbonyl)-benzamide-hydrochloride

a. 3-(1-amino-ethyl)-4-benzyloxy-benzonitrile

10.4 g (41.4 mmol) of 3-acetyl-4-benzoyl-benzonitrile are dissolved in 40 ml methanol, combined with 31.0 g (0.4 mol) of ammonium acetate and, after the addition of 1.8 g (28 mmol) of sodium cyanoborohydride, refluxed for 3 days under a nitrogen atmosphere. The solvent is distilled off, the residue is stirred in semiconc. hydrochloric acid for 30 minutes, neutralised with ammonia and extracted with ethyl acetate. The combined organic extracts are evaporated down and chromatographed on silica gel, eluting with petroleum ether/ethanol 9:1 and with ethyl acetate/ethanol 7:3.

Yield: 1.3 g (12% of theoretical),

R_{f} value: 0.15 (silica gel; ethyl acetate/ethanol=9:1)

b. N-[1-(2-benzyloxy-5-cyano-phenyl)-ethyl]-3-methyl-4-(pyrrolidin-1-yl-carbonyl)-benzamide

Prepared analogously to Example 1.a. from 3-methyl-4-(pyrrolidin-1-yl-carbonyl)-benzoic acid, 3-(1-amino-ethyl)-4-benzyloxy-benzonitrile, O-(benzotriazol-1-yl)-N,N,N′,N′-tetramethyl-uronium tetrafluoroborate and N-methylmorpholine in dimethylformamide.

Yield: 63% of theoretical,

R_{f} value: 0.45 (silica gel; dichloromethane/ethanol=19:1)

c. N-[1-(5-carbamimidoyl-2-hydroxy-phenyl)-ethyl]-3-methyl-4-(pyrrolidin-1-yl-carbonyl)-benzamide-hydrochloride

Prepared analogously to Example 1.g. from N-[1-(2-benzyloxy-5-cyano-phenyl)-ethyl]-3-methyl-4-(pyrrolidin-1-yl-carbonyl)-benzamide and hydrochloric acid/ammonium carbonate in ethanol followed by reaction with hydrogen/palladium on activated charcoal in methanol analogously to Example 3.g.

Yield: 30% of theoretical,

R_{f} value: 0.35 (Reversed Phase RP 8; 5% sodium chloride solution/methanol 2:3)

C₂₂H₂₆N₄O₃xHCl (394.48/430.95)

Mass spectrum: (M+H)⁺=395 (M−H)⁻=393 (M+Cl)⁻=429/31 (C1)

The following compounds are prepared analogously to Example 15:

(1) N-[1-(5-carbamimidoyl-2-hydroxy-phenyl)-ethyl]-3-bromo-4-(pyrrolidin-1-yl-carbonyl)-benzamide-hydrochloride

Yield: 3% of theoretical

R_{f} value: 0.50 (Reversed Phase RP 8; 5% sodium chloride solution/methanol=2:3)

C₂₁H₂₃BrN₄O₃xHCl (459.35/495.82)

Mass spectrum: (M+H)⁺=459/61 (Br)

(2) N-[1-(5-carbamimidoyl-2-hydroxy-phenyl)-ethyl]-4-(pyrrolidin-1-yl-carbonyl)-benzamide-hydrochloride

Yield: 5% of theoretical

R_{f} value: 0.58 (Reversed Phase RP 8; 5% sodium chloride solution/methanol=2:3) C₂₁H₂₄N₄O₃xHCl (380.45/416.92)

Mass spectrum: (M+H)⁺=381

### EXAMPLE 16

Ethyl 3-(3-carbamimidoyl-phenyl)-3-[3-trifluoromethyl-4-(pyrrolidin-1-yl-carbonyl)-benzoylamino]-propionate-hydrochloride

a. 4-methyl-3-trifluoromethyl-benzonitrile

10.0 g (57 mmol) of 4-methyl-3-trifluoromethyl-aniline are suspended in 50 ml of semiconcentrated hydrochloric acid and at 0° C. combined with a solution of 4.2 g (61 mmol) of sodium nitrite in 25 ml of water. The diazonium salt solution formed is then added dropwise at 30° C. to a solution of 12.5 g (0.14 mol) of copper-(I)-cyanide and 25 g (0.38 mol) of potassium cyanide in 160 ml of water. The suspension formed is heated to 78° C. for 2 hours. After cooling the undissolved material is filtered off, the filtrate is combined with 250 ml ethyl acetate and extracted. The combined organic phases are washed until neutral, dried and evaporated down. The crude product is purified by sublimation at 40-90° C. and 12 mbar.

Yield: 5.5 g (47% of theoretical),

R_{f} value: 0.43 (silica gel; cyclohexane/ethyl acetate=4:1)

b. 4-cyano-2-trifluoromethyl-benzoic acid/4-aminocarbonyl-2-trifluoromethyl-benzoic acid

28 ml of conc. sulphuric acid are added dropwise at ambient temperature to a suspension of 4.5 g (24.3 mmol) of 4-methyl-3-trifluoromethyl-benzonitrile and 11 g (37.4 mmol) of potassium dichromate in 100 ml glacial acetic acid, while the temperature rises to 80° C. After 1.5 hours at 115° C. the reaction mixture is cooled, poured onto a mixture of 300g of ice/300 ml of saturated saline solution, adjusted to pH 3.5 with conc. ammonia and extracted with a total of 1000 ml of ethyl acetate. The combined organic phases are evaporated down by half, adjusted to pH 9 with conc. ammonia and extracted with a total of 400 ml of 0.1 N sodium hydroxide solution. The aqueous phase is adjusted to pH 3.5 by the addition of conc. hydrochloric acid and extracted with a total of 400 ml of ethyl acetate. The combined organic extracts are washed with saturated saline solution, dried and evaporated down.

Yield: 2.6 g (product mixture in a ratio of 3:7, 47% of theoretical),

R_{f} values:

4-cyano-2-trifluoromethyl-benzoic acid: 0.3 (silica gel; methylene chloride/ethanol=6.5 3.5+glacial acetic acid)

4-aminocarbonyl-2-trifluoromethyl-benzoic acid: 0.2 (silica gel; methylene chloride/ethanol=6.5:3.5+glacial acetic acid)

c. 3-trifluoromethyl-4-(pyrrolidin-1-yl-carbonyl)-benzonitrile/3-trifluoromethyl-4-(pyrrolidin-1-yl-carbonyl)-benzamide

Prepared analogously to Example 1.a. from 4-cyano-2-trifluoromethyl-benzoic acid/4-aminocarbonyl-2-trifluoromethyl-benzoic acid, pyrrolidine, O-(benzotriazol-1-yl)-N,N,N′,N′-tetramethyluronium tetrafluoroborate and N-methylmorpholine in dimethylformamide.

Yield: 53% of theoretical (product mixture),

R_{f} values:

3-trifluoromethyl-4-(pyrrolidin-1-yl-carbonyl)-benzonitrile: 0.35 (silica gel; ethyl acetate/ethanol=85:15+glacial acetic acid)

3-trifluoromethyl-4-(pyrrolidin-1-yl-carbonyl)-benzamide: 0.35 (silica gel; ethyl acetate/ethanol=85:15+glacial acetic acid)

d. 3-trifluoromethyl-4-(pyrrolidin-1-yl-carbonyl)-benzoic acid

1.65 g of a mixture of 3-trifluoromethyl-4-(pyrrolidin-1-yl-carbonyl)-benzonitrile and 3-trifluoromethyl-4-(pyrrolidin-1-yl-carbonyl)-benzamide are dissolved in 20 ml ethanol and combined with 20 ml of 10 N sodium hydroxide solution. After 45 minutes at 80° C. the reaction solution is poured onto ice water and adjusted to pH 9 with conc. hydrochloric acid. The ethanol is distilled off and the residue is extracted with 100 ml ether and 50 ml ethyl acetate. The aqueous phase is adjusted to pH 3.5 with conc. hydrochloric acid, the white precipitate formed is suction filtered and dried.

Yield: 1.05 g (85% of theoretical),

R_{f} value: 0.43 (silica gel; ethyl acetate/ethanol=85:15+glacial acetic acid)

e. ethyl 3-(3-cyano-phenyl)-3-[3-trifluoromethyl-4-(pyrrolidin-1-yl-carbonyl)-benzoylamino]-propionate

Prepared analogously to Example 1.a. from 3-trifluoromethyl-4-(pyrrolidin-1-yl-carbonyl)-benzoic acid, O-(benzotriazol-1-yl)-N,N,N′,N′-tetramethyluronium tetrafluoroborate, N-methylmorpholine and ethyl 3-amino-3-(3-cyano-phenyl)-propionate in dimethylformamide.

Yield: 64% of theoretical,

R_{f} value: 0.7 (silica gel; ethyl acetate/ethanol=9:1)

f. ethyl 3-(3-carbamimidoyl-phenyl)-3-[3-trifluoromethyl-4-(pyrrolidin-1-yl-carbonyl)-benzoylamino]-propionate-hydrochloride

Prepared analogously to Example 1.g. from ethyl 3-(3-cyano-phenyl)-3-[3-trifluoromethyl-4-(pyrrolidin-1-yl-carbonyl)-benzoylamino]-propionate and hydrochloric acid/ammonium carbonate in ethanol.

Yield: 90% of theoretical,

R_{f} value: (Reversed Phase RP 8; 5% sodium chloride solution/methanol=2:3)

C₂₅H₂₇F₃N₄O₄xHCl (504.51/540.97)

Mass spectrum: (M+H)⁺=505

### EXAMPLE 17

N-(5-carbamimidoyl-2-hydroxy-benzyl)-3-trifluoromethoxy-4-(pyrrolidin-1-yl-carbonyl)-benzamide-hydrochloride

a. 3-trifluoromethoxy-4-(pyrrolidin-1-yl-carbonyl)-benzoic acid

1.8 g of carbonyldiimidazole (15.6 mmol) and 2.5 ml of N-methylmorpholine (22.7 mmol) are added to a solution of 2.5 g of 2-(trifluoromethoxy)-terephthalic acid (19 mmol) in 40 ml of dimethylformamide at ambient temperature. After 10 minutes 1.3 ml of pyrrolidine (15.6 mmol) are added dropwise. The reaction mixture is stirred for 3 days at ambient temperature, then stirred into ice water, adjusted to pH 4 with 1 N hydrochloric acid and extracted 3× with 100 ml of ethyl acetate. The combined organic phases are washed with saline solution, dried and evaporated down. The crude product is purified on silica gel, eluting initially with dichloromethane, then with dichloromethane/ethanol 50:1, 25:1, 19:1 and 9:1. The uniform fractions are combined and evaporated down.

Yield: 90 mg (3% of theoretical),

R_{f} value: 0.27 (silica gel; dichloromethane/ethanol=9:1)

b. N-(2-benzyloxy-5-cyano-benzyl)-3-trifluoromethoxy-4-(pyrrolidin-1-yl-carbonyl)-benzamide

Prepared analogously to Example 1.a. from 3-trifluoromethyl-4-(pyrrolidin-1-carbonyl)-benzoic acid, O-(benzotriazol-1-yl)-N,N,N′,N′-tetramethyluronium tetrafluoroborate, N-methylmorpholine and 4-benzyloxy-3-aminomethyl-benzonitrile in dimethylformamide.

Yield: 62% of theoretical,

R_{f} value: 0.5 (silica gel; dichloromethane/ethanol=9:1)

c. N-(5-carbamimidoyl-2-hydroxy-benzyl)-3-trifluoromethoxy-4-(pyrrolidin-1-yl-carbonyl)-benzamide-hydrochloride

Prepared analogously to Example 1.g. from N-(2-benzyloxy-5-cyano-benzyl)-3-trifluoromethoxy-4-(pyrrolidin-1-yl-carbonyl)-benzamide and hydrochloric acid/ammonium carbonate in ethanol followed by reaction with hydrogen/palladium on activated charcoal in methanol analogously to Example 3.g.

Yield: 24% of theoretical,

R_{f} value: 0.3 (Reversed Phase RP 8; 5% sodium chloride solution/methanol=2:3)

C₂₁H₂₁F₃N₄O₄xHCl (450.42/486.88)

Mass spectrum: (M+H)⁺=451 (M−H)⁻=449

### EXAMPLE 18

3-(5-carbamimidoyl-2-hydroxy-phenyl)-3-[3-methyl-4-(pyrrolidin-1-yl-carbonyl)-benzoylamino]-propionic acid-hydrochloride

a. 4-amino-2-oxo-chroman-6-carbonitrile-hydrochloride

4.6 ml of a 1 N solution of bis-(trimethylsilyl)-lithium amide in tetrahydrofuran (4.6 mmol) are added dropwise to a solution of 750 mg of 2-oxo-2H-chromene-6-carbonitrile (4.4 mmol) at −70° C. After 5 minutes at −70° C. and 2 hours at −15° C. the reaction mixture is poured onto 180 ml of diethylether and combined with ethereal hydrochloric acid. The precipitate formed is filtered off, dried and further reacted without purification.

Yield: 1.0 g (56% of theoretical),

R_{f} value: 0.45 (silica gel; ethyl acetate/ethanol=9:1+1% ammonia))

b. 3-(5-cyano-2-hydroxy-phenyl)-3-[3-methyl-4-(pyrrolidin-1-yl-carbonyl)-benzoyl-amino]-propionic acid

Prepared analogously to Example 12.b. from 4-amino-2-oxo-chromane-6-carbonitrile-hydrochloride, 3-methyl-4-(pyrrolidin-1-carbonyl)-benzoylchloride and triethylamine in tetrahydrofuran.

Yield: 39% of theoretical,

R_{f} value: 0.5 (silica gel; ethyl acetate/ethanol=4:1+1% glacial acetic acid)

c. ethyl 3-(5-carbamimidoyl-2-hydroxy-phenyl)-3-[3-methyl-4-(pyrrolidin-1-yl-carbonyl)-benzoylamino]-propionate-hydrochloride

Prepared analogously to Example 1.g. from 3-(5-cyano-2-hydroxy-phenyl)-3-[3-methyl-4-(pyrrolidin-1-yl-carbonyl)-benzoylamino]-propionic acid and hydrochloric acid/ammonium carbonate in ethanol.

Yield: 69% of theoretical,

R_{f} value: 0.5 (Reversed Phase RP 8; 5% sodium chloride solution/methanol=2:3)

d. 3-(5-carbamimidoyl-2-hydroxy-phenyl)-3-[3-methyl-4-(pyrrolidin-1-yl-carbonyl)-benzoylamino]-propionic acid-hydrochloride

Prepared analogously to Example 11. from ethyl 3-(5-carbamimidoyl-2-hydroxy-phenyl)-3-[3-methyl-4-(pyrrolidin-1-yl-carbonyl)-benzoylamino]-propionate hydrochloride and 6 N hydrochloric acid.

Yield: 43% of theoretical,

R_{f} value: 0.6 (Reversed Phase RP 8; 5% sodium chloride solution/methanol 2:3)

C₂₃H₂₆N₄O₅xHCl (438.48/474.95)

Mass spectrum: (M+H)⁺=439

### EXAMPLE 19

Ethyl 3-[3-N-(phenylcarbonyl)-amidino-phenyl]-3-[3-methyl-4-(pyrrolidin-1-yl-carbonyl)-benzoylamino]-propionate

A suspension of 390 mg (0.8 mmol) of ethyl 3-(3-carbamimidoyl-phenyl)-3-[3-methyl-4-(pyrrolidin-1-yl-carbonyl)-benzoylamino]-propionate and 1.0 ml triethylamine in 40 ml dichloromethane is combined with 275 mg (1.1 mmol) of 4-nitrophenyl benzoate and refluxed for 7 hours. The solvent is evaporated off, the residue is taken up in ice water and adjusted to pH 4 with 1N hydrochloric acid. After extraction with ethyl acetate the combined organic phases are washed with saline solution and dried. The crude product is purified on silica gel, eluting initially with dichloromethane, then with dichloromethane/ethanol 50:1 and 25:1.

Yield: 55 mg (12% of theoretical),

R_{f} value: 0.45 (silica gel; dichloromethane/ethanol=19:1)

C₃₂H₃₄N₄O₅ (554.65)

Mass spectrum: (M+H)⁺=555 (M−H)⁻=553

The following compounds are prepared analogously to Example 19:

(1) ethyl 3-[3-N-(n-hexyloxycarbonyl)-amidino-phenyl]-3-[3-methyl-4-(pyrrolidin-1-yl-carbonyl)-benzoylamino]-propionate

Yield: 47% of theoretical,

R_{f} value: 0.45 (silica gel; dichloromethane/ethanol=19:1+1% ammonia)

C₃₂H₄₂N₄O₆xHCl (578.71/615.18)

Mass spectrum: (M+H)⁺=579

(2) n-propyl 3-[3-N-(phenylcarbonyl)-amidino-phenyl]-3-[3-methyl-4-(pyrrolidin-1-yl-carbonyl)-benzoylamino]-propionate

Yield: 23% of theoretical,

R_{f} value: 0.70 (silica gel; ethyl acetate/ethanol=9:1)

C₃₃H₃₆N₄O₅ (568.68)

Mass spectrum: (M+H)⁺=569 (M−H)⁻=567

(3) ethyl 3-[3-N-(2,2,2-trichloroethyloxycarbonyl)-amidino-phenyl]-3-[3-methyl-4-(pyrrolidin-1-yl-carbonyl)-benzoylamino]-propionate

Yield: 45% of theoretical,

R_{f} value: 0.70 (silica gel; ethyl acetate/ethanol=9:1)

C₂₈H₃₁Cl₃N₄O₆ (625.94)

Mass spectrum: (M+H)⁺=625/7/9 (Cl₃) (M−H)⁻=623/5/7 (Cl₃) (M+HCOO)⁻=669/71/71 (Cl₃)

(4) N-{5-[N-(n-hexyloxycarbonyl)-amidino]-2-hydroxy-benzyl}-3-methyl-4-(pyrrolidin-1-yl-carbonyl)-benzamide

Yield: 11% of theoretical,

R_{f} value: 0.50 (silica gel; ethyl acetate/ethanol=9:1)

C₂₈H₃₆N₄O₅ (508.62)

Mass spectrum: (M+H)⁺=509 (M−H)⁻=507

(5) N— {5-[N-(phenylcarbonyl)-amidino]-2-hydroxy-benzyl}-3-methyl-4-(pyrrolidin-1-yl-carbonyl)-benzamide

Yield: 46% of theoretical,

R_{f} value: 0.45 (silica gel; ethyl acetate/ethanol 9:1)

C₂₈H₂₈N₄O₄ (484.56)

Mass spectrum: (M+H)⁺=585 (M−H)⁻=583

### EXAMPLE 20

N-[5-(N-hydroxy-amidino)-2-hydroxy-benzyl]-3-methyl-4-(pyrrolidin-1-yl-carbonyl)-benzamide-hydrochloride

a. N-(5-N-hydroxyamidino-2-benzyloxy-benzyl)-3-methyl-4-(pyrrolidin-1-yl-carbonyl)-benzamide

A solution of 482 mg (1.06 mmol) of N-(2-benzyloxy-5-cyano-benzyl)-3-methyl-4-(pyrrolidin-1-yl-carbonyl)-benzamide in 20 ml methanol/ethanol (1:1) is combined with a solution of 148 mg (2.1 mmol) of hydroxylamine hydrochloride and 174 mg (2.1 mmol) of sodium acetate in 1.0 ml of water and refluxed for 7 hours. After cooling the reaction mixture is combined with ice water and extracted with ethyl acetate. The combined organic phases are washed with saline solution, dried and evaporated down. The crude product is purified on silica gel, eluting initially with dichloromethane, then with dichloromethane/ethanol 25:1, 19:1 and 9:1.

Yield: 210 mg (41% of theoretical),

R_{f} value: 0.40 (silica gel; dichloromethane/ethanol=19:1)

b. N-(5-N-hydroxyamidino-2-hydroxy-benzyl)-3-methyl-4-(pyrrolidin-1-yl-carbonyl)-benzamide-hydrochloride

Prepared analogously to Example 3.g. from N-(5-N-hydroxyamidino-2-benzyloxy-benzyl)-3-methyl-4-(pyrrolidin-1-yl-carbonyl)-benzamide and hydrogen/palladium on activated charcoal.

Yield: 41% of theoretical,

R_{f} value: 0.3 (Reversed Phase RP 8; 5% sodium chloride solution/methanol=2:3)

C₂₁H₂₄N₄O₄xHCl (396.45/432.91)

Mass spectrum: (M+H)⁺=397 (M−H)⁻=395

### EXAMPLE 21

N-{5-[N-(phenylcarbonyl)-amidino]-2-(ethyloxycarbonyloxy)-benzyl}-3-methyl-4-(pyrrolidin-1-yl-carbonyl)-benzamide

121 mg (0.25 mmol) of N-(5-phenylcarbonylamidino-2-hydroxy-benzyl)-3-methyl-4-(pyrrolidin-1-yl-carbonyl)-benzamide are dissolved in 10 ml isopropanol with heating and combined with a solution of 41.5 mg (0.3 mmol) of potassium carbonate in 0.5 ml of water. After 10 minutes a solution of 32.6 mg (0.3 mmol) of ethyl chloroformate in 1 ml of isopropanol is added. After 1 hour at ambient temperature the reaction solution is stirred into ice water. The precipitate formed is suction filtered, washed with water and dried.

Yield: 72 mg (52% of theoretical),

R_{f} value: 0.50 (silica gel; dichloromethane/ethanol=9:1)

C₃₁H₃₂N₄O₆ (556.62)

Mass spectrum: (M+H)⁺=557 (M−H)⁻=555

### EXAMPLE 22

Dry ampoule containing 75 mg of active substance per 10 ml

Composition:

| | | |
|---|---|---|
| Active substance | 75.0 | mg |
| Mannitol | 50.0 | mg |
| water for injections | ad 10.0 | ml |

Preparation:

Active substance and mannitol are dissolved in water. After packaging the solution is freeze-dried. To produce the solution ready for use, the product is dissolved in water for injections.

### EXAMPLE 23

Dry ampoule containing 35 mg of active substance per 2 ml

Composition:

| | | |
|---|---|---|
| Active substance | 35.0 | mg |
| Mannitol | 100.0 | mg |
| water for injections | ad 2.0 | ml |

Preparation:

Active substance and mannitol are dissolved in water. After packaging, the solution is freeze-dried.

To produce the solution ready for use, the product is dissolved in water for injections.

### EXAMPLE 24

Tablet containing 50 mg of active substance

Composition:

| | | |
|---|---|---|
| (1) Active substance | 50.0 | mg |
| (2) Lactos | 98.0 | mg |
| (3) Maize starch | 50.0 | mg |
| (4) Polyvinylpyrrolidone | 15.0 | mg |
| (5) Magnesium stearate | 2.0 | mg |
| | 215.0 | mg |

Preparation:

(1), (2) and (3) are mixed together and granulated with an aqueous solution of (4). (5) is added to the dried granulated material. From this mixture tablets are pressed, biplanar, faceted on both sides and with a dividing notch on one side.

Diameter of the tablets: 9 mm.

### EXAMPLE 25

Tablet containing 350 mg of active substance

Composition:

| | | |
|---|---|---|
| (1) Active substance | 350.0 | mg |
| (2) Lactose | 136.0 | mg |
| (3) Maize starch | 80.0 | mg |
| (4) Polyvinylpyrrolidone | 30.0 | mg |
| (5) Magnesium stearate | 4.0 | mg |
| | 600.0 | mg |

Preparation:

(1), (2) and (3) are mixed together and granulated with an aqueous solution of (4). (5) is added to the dried granulated material. From this mixture tablets are pressed, biplanar, faceted on both sides and with a dividing notch on one side. Diameter of the tablets: 12 mm.

### EXAMPLE 26

Capsules containing 50 mg of active substance

Composition:

| | | |
|---|---|---|
| (1) Active substance | 50.0 | mg |
| (2) Dried maize starch | 58.0 | mg |
| (3) Powdered lactose | 50.0 | mg |
| (4) Magnesium stearate | 2.0 | mg |
| | 160.0 | mg |

Preparation:

(1) is triturated with (3). This trituration is added to the mixture of (2) and (4) with vigorous mixing.

This powder mixture is packed into size 3 hard gelatine capsules in a capsule filling machine.

### EXAMPLE 27

Capsules containing 350 mg of active substance

Composition:

| | | |
|---|---|---|
| (1) Active substance | 350.0 | mg |
| (2) Dried maize starch | 46.0 | mg |
| (3) Powdered lactose | 30.0 | mg |
| (4) Magnesium stearate | 4.0 | mg |
| | 430.0 | mg |

Preparation:

(1) is triturated with (3). This trituration is added to the mixture of (2) and (4) with vigorous mixing.

This powder mixture is packed into size 0 hard gelatine capsules in a capsule filling machine.

### EXAMPLE 28

Suppositories containing 100 mg of active substance 1 suppository contains:

| | | |
|---|---|---|
| Active substance | 100.0 | mg |
| Polyethyleneglycol (M.W. 1500) | 600.0 | mg |
| Polyethyleneglycol (M.W. 6000) | 460.0 | mg |
| Polyethylenesorbitan monostearate | 840.0 | mg |
| | 2,000.0 | mg |

Preparation:

The polyethyleneglycol is melted together with polyethylenesorbitan monostearate. At 40° C. the ground active substance is homogeneously dispersed in the melt. This is then cooled to 38° C. and poured into slightly chilled suppository moulds.

What is claimed is:

1. A compound of the formula 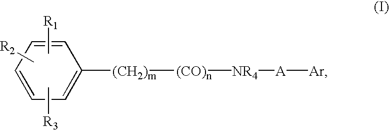
wherein:
(i) m denotes the number 0,
n denotes the number 1 and
A denotes a straight-chain C₁₋₃-alkylene group wherein
one or two hydrogen atoms independently of one another may be replaced in each case by a C₁₋₃-alkyl group or
a hydrogen atom may be replaced by the group —(CH₂)ₚ—R_{f}, while
p denotes one of the numbers 0, 1, 2 or 3 and
R_{f} denotes a hydroxycarbonyl, C₁₋₃-alkoxycarbonyl, aminocarbonyl, C₁₋₃-alkylaminocarbonyl, di-(C₁₋₃-alkyl)-aminocarbonyl, C₃₋₇-cycloalkylamino-carbonyl, N-(C₁₋₃-alkoxy-carbonylmethyl)-N-(C₁₋₃-alkyl)-aminocarbonyl, N-(carboxymethyl)-N-(C₁₋₃-alkyl)-aminocarbonyl or a 4- to 7-membered cycloalkyleneimino-carbonyl group, or
(ii) m denotes the number 1,
n denotes the number 1 and
A denotes a bond or
(iii) m denotes the number 0 or 1,
n denotes the number 0 and
A denotes a straight-chain C₁₋₃-alkylene group wherein one or two hydrogen atoms independently of one another may be replaced in each case by a C₁₋₃-alkyl group, or
(iv) m denotes the number 2,
n denotes the number 0 and
A denotes a bond,
R₁ denotes an amino, C₁₋₅-alkylamino, C₃₋₇-cycloalkylamino or phenyl-C₁₋₃-alkylamino group each of which may be substituted at the amino nitrogen atom by a phenylcarbonyl or phenylsulphonyl group or by a C₁₋₃-alkyl or C₁₋₃-alkyl-carbonyl group optionally substituted in the alkyl moiety by a carboxy group, a group which may be converted in vivo into a carboxy group, an amino, C₁₋₃-alkylamino or di-(C₁₋₃-alkyl)-amino group,
a di-(C₁₋₅-alkyl)amino or N-(C₃₋₇-cycloalkyl)-C₁₋₅-alkylamino group, while the C₁₋₅-alkyl moiety with the exception of the 1 position may be substituted in each case by a hydroxy, C₁₋₃-alkoxy, amino, C₁₋₃-alkyl-amino or di-(C₁₋₃-alkyl)-amino group,
a 4- to 7-membered cycloalkyleneiminocarbonyl or cycloalkyleneiminosulphonyl group optionally substituted by a C₁₋₃-alkyl, amino-C₁₋₃-alkyl, C₁₋₃-alkylamino-C₁₋₃-alkyl, di-(C₁₋₃-alkyl)-amino-C₁₋₃-alkyl, aminocarbonyl, C₁₋₃-alkylamino-carbonyl or di-(C₁₋₃-alkyl)-aminocarbonyl group,
a 2,5-dihydropyrrol-1-yl-carbonyl group,
an aminosulphonyl group optionally substituted by one or two C₁₋₃-alkyl groups,
a C₃₋₇-cycloalkyl-carbonyl group, whilst
the methylene group in the 3 or 4 position in a C₅₋₇-cycloalkyl-carbonyl group may be replaced by an —NH group wherein
the hydrogen atom of the —NH group may be replaced by a C₁₋₃-alkyl, C₁₋₃-alkyl-carbonyl, phenylcarbonyl or phenylsulphonyl group,
a phenylcarbonyl or heteroarylcarbonyl group,
or a C₁₋₃-alkyl group optionally monosubstituted by an amino, C₁₋₃-alkylamino, di-(C₁₋₃-alkyl)-amino, hydroxy, phenyl or a 4- to 7-membered cycloalkyleneimino group or terminally disubstituted by a phenyl group and a hydroxy group, while
the phenyl substituents may be substituted by an amidino group optionally substituted by one or two C₁₋₃-alkyl groups, by a fluorine, chlorine or bromine atom, by a trifluoromethyl, C₁₋₃-alkyl or C₁₋₃-alkoxy group,
R₂ denotes a hydrogen, fluorine, chlorine or bromine atom, a C₁₋₃-alkyl group wherein the hydrogen atoms may be wholly or partly replaced by fluorine atoms, a C₂₋₃-alkenyl, C₂₋₃-alkynyl, hydroxy, C₁₋₃-alkoxy or trifluoromethoxy group,
R₃ denotes a hydrogen atom or a C₁₋₃-alkyl group,
R₄ denotes a hydrogen atom or a C₁₋₃-alkyl group optionally substituted by a carboxy group or a group which may be converted in vivo into a carboxy group and
Ar denotes a phenyl or naphthyl group substituted by the groups R₅, R₆ and R₇, while
R₅ denotes a cyano group, an amidino group optionally substituted by one or two C₁₋₃-alkyl groups, an amino-C₁₋₃-alkyl, C₁₋₃-alkylamino-C₁₋₃-alkyl or di-(C₁₋₃-alkyl)amino-C₁₋₃-alkyl group,
R₆ denotes a hydrogen, fluorine, chlorine or bromine atom, a trifluoromethyl, C₁₋₃-alkyl, hydroxy, hydroxy-C₁₋₃-alkyl, C₁₋₃-alkoxy, C₁₋₃-alkoxy-C₁₋₃-alkyl, carboxy, carboxy-C₁₋₃-alkyl, carboxy-C₁₋₃-alkoxy, C₁₋₄-alkoxy-carbonyloxy, C₁₋₄-alkoxy-carbonyl-C₁₋₃-alkoxy, phenyl-C₁₋₃-alkoxy, amino, C₁₋₃-alkylamino or di-(C₁₋₃-alkyl)amino group and
R₇ denotes a hydrogen, fluorine, chlorine or bromine atom or a C₁₋₃-alkyl group,
or a thienyl, thiazolyl, pyridinyl, pyrimidinyl, pyrazinyl or pyridazinyl group optionally substituted in the carbon skeleton by a C₁₋₃-alkyl group,
while the term heteroaryl group mentioned above denotes a 5-membered heteroaryl group bound via a carbon or nitrogen atom which contains
an imino group optionally substituted by a C₁₋₄-alkyl or C₁₋₄-alkyl-carbonyl group, an oxygen or sulphur atom,
an imino group optionally substituted by a C₁₋₄-alkyl group or an oxygen or sulphur atom and additionally a nitrogen atom,
an imino group optionally substituted by a C₁₋₄-alkyl group and two nitrogen atoms or
an oxygen or sulphur atom and two nitrogen atoms,
or a 6-membered heteroaryl group which contains one or two nitrogen atoms,
while a phenyl ring may be fused to the abovementioned 5- or 6-membered heteroaryl groups via two adjacent carbon atoms and the bicyclic heteroaryl groups thus formed may be bound via the heteroaromatic or carbocyclic moiety, and the unsubstituted or monosubstituted phenyl groups mentioned in the definition of the abovementioned groups, or the unsubstituted or monosubstituted phenyl moieties contained in these groups, as well as the abovementioned heteroaryl groups may additionally be substituted at a carbon atom in each case by a fluorine, chlorine or bromine atom, by a trifluoromethyl, C₁₋₃-alkyl or C₁₋₃-alkoxy group, unless otherwise stated,
the carboxy groups mentioned in the definition of the abovementioned groups may be replaced by a group which may be converted in vivo into a carboxy group or by a group which is negatively charged under physiological conditions, and
the amino and imino groups mentioned in the definition of the abovementioned groups may be substituted by a group which can be cleaved in vivo,
or a salt thereof.

2. A compound of the formula I according to claim 1, wherein:
(i) m denotes the number 0,
n denotes the number 1 and
A denotes a straight-chain C₁₋₃-alkylene group wherein
one or two hydrogen atoms independently of one another may be replaced in each case by a C₁₋₃-alkyl group or
a hydrogen atom may be replaced by the group —(CH₂)ₚ—R_{f}, while
p denotes one of the numbers 0, 1, 2 or 3 and
R_{f} denotes a hydroxycarbonyl, C₁₋₃-alkoxycarbonyl, aminocarbonyl, C₁₋₃-alkylaminocarbonyl, di-(C₁₋₃-alkyl)-aminocarbonyl, C₃₋₇-cycloalkylamino-carbonyl, N-(C₁₋₃-alkoxy-carbonylmethyl)-N-(C₁₋₃-alkyl)-aminocarbonyl, N-(carboxymethyl)-N-(C₁₋₃-alkyl)-aminocarbonyl or a 4- to 7-membered cycloalkyleneimino-carbonyl group, or
(ii) m denotes the number 0 or 1,
n denotes the number 0 and
A denotes a straight-chain C₁₋₃-alkylene group wherein one or two hydrogen atoms independently of one another may be replaced in each case by a C₁₋₃-alkyl group,
R₁ denotes an amino, C₁₋₃-alkylamino or C₃₋₇-cycloalkylamino group each of which may be substituted at the amino nitrogen atom by a C₁₋₃-alkyl, C₁₋₃-alkylcarbonyl, carboxy-C₁₋₃-alkyl, carboxy-C₁₋₃-alkylcarbonyl, C₁₋₆-alkoxy-carbonyl-C₁₋₃-alkyl-carbonyl or amino-C₁₋₃-alkyl-carbonyl group,
a di-(C₁₋₃-alkyl)amino or N-(C₅₋₇-cycloalkyl)-C₁₋₃-alkylamino group,
a 4- to 7-membered cycloalkyleneiminocarbonyl group optionally substituted by a C₁₋₃-alkyl, amino-C₁₋₃-alkyl, C₁₋₃-alkylamino-C₁₋₃-alkyl, aminocarbonyl or C₁₋₃-alkylamino-carbonyl group, while
a hydrogen atom bound to a nitrogen atom may be replaced by an acetyl, phenylcarbonyl or tert.-butoxycarbonyl group, or a 2,5-dihydropyrrol-1-yl-carbonyl group,
R₂ denotes a hydrogen, fluorine, chlorine or bromine atom, a C₁₋₃-alkyl, C₂₋₃-alkenyl, C₂₋₃-alkynyl, trifluoromethyl, C₁₋₃-alkoxy or trifluoromethoxy group,
R₃ denotes a hydrogen atom or a C₁₋₃-alkyl group,
R₄ denotes a hydrogen atom or a C₁₋₃-alkyl group and
Ar denotes a phenyl group substituted by the groups R₅, R₆ and R₇, while
R₅ denotes a cyano group, an amidino group optionally substituted by one or two C₁₋₃-alkyl groups, a hydroxy, C₁₋₆-alkoxy-carbonyl, 2,2,2-trichloroethoxycarbonyl or phenylcarbonyl group, or an amino-C₁₋₃-alkyl or C₁₋₃-alkylamino-C₁₋₃-alkyl group,
R₆ denotes a hydrogen, fluorine, chlorine or bromine atom, a trifluoromethyl, C₁₋₃-alkyl, hydroxy, hydroxy-C₁₋₃-alkyl, C₁₋₃-alkoxy, carboxy, C₁₋₃-alkoxy-carbonyloxy, carboxy-C₁₋₃-alkoxy or C₁₋₄-alkoxy-carbonyl-C₁₋₃-alkoxy group and
R₇ denotes a hydrogen atom or a C₁₋₃-alkyl group,
while the unsubstituted or monosubstituted phenyl groups mentioned in the definition of the abovementioned groups, or the unsubstituted or monosubstituted phenyl moieties contained in these groups, as well as the abovementioned heteroaryl groups may additionally be substituted at a carbon atom in each case by a fluorine, chlorine or bromine atom, by a trifluoromethyl, C₁₋₃-alkyl or C₁₋₃-alkoxy group, unless otherwise stated,
or a salt thereof.

3. A compound of the formula I according to claim 2, wherein:
(i) m denotes the number 0,
n denotes the number 1 and
A denotes a methylene group wherein
one or two hydrogen atoms independently of one another may be replaced in each case by a C₁₋₃-alkyl group or
a hydrogen atom may be replaced by the group —(CH₂)ₚ—R_{f}, while
p denotes one of the numbers 0, 1, 2 or 3 and
R_{f} denotes a hydroxycarbonyl, C₁₋₃-alkoxycarbonyl, N-(C₁₋₃-alkyl)-amino-carbonyl, di-(C₁₋₃-alkyl)-aminocarbonyl, N-(C₁₋₃-alkoxy-carbonylmethyl)-N-(C₁₋₃-alkyl)-aminocarbonyl, N-(carboxymethyl)-N-(C₁₋₃-alkyl)-aminocarbonyl or a 4- to 7-membered cycloalkyleneimino-carbonyl group or
(ii) m denotes the number 0,
n denotes the number 0 and
A denotes a —CH₂—CH₂— group, or
(iii) m denotes the number 1,
n denotes the number 0 and
A denotes a —CH₂— group,
the groups R₁ to R₄ are defined as in claim 2, but R₁ in the 4 position is bound to the phenyl group contained in formula I and
Ar denotes a phenyl group disubstituted by the groups R₅ and R₆, while
R₅ is bound in the 3 position if R₆ denotes a hydrogen atom, or is bound in the 5 position if R₆ assumes a meaning other than the hydrogen atom, and denotes an amidino group optionally substituted by one or two C₁₋₃-alkyl groups, a hydroxy, C₁₋₆-alkoxy-carbonyl, 2,2,2-trichloroethoxycarbonyl or phenylcarbonyl group, or an amino-C₁₋₃-alkyl or C₁₋₃-alkylamino-C₁₋₃-alkyl group and
R₆ denotes a hydrogen atom or a hydroxy, C₁₋₃-alkoxy, carboxy-C₁₋₃-alkoxy, C₁₋₃-alkoxy-carbonyloxy- or C₁₋₄-alkoxy-carbonyl-C₁₋₃-alkoxy group bound in the 2 position,
or a salt thereof.

4. A compounds of the formula I according to claim 1, wherein:
(i) m denotes the number 0,
n denotes the number 1 and
A denotes a methylene group wherein a hydrogen atom may be replaced by a methyl, hydroxycarbonyl, C₁₋₃-alkoxy-carbonyl, C₁₋₃-alkylaminocarbonyl, dimethylaminocarbonyl, pyrrolidin-1-yl-carbonyl, C₁₋₃-alkylaminocarbonylmethyl, N-(hydroxy-carbonyl-methyl)-N-(C₁₋₃-alkyl)-amino-carbonyl-methyl, N-(C₁₋₃-alkoxy-carbonyl-methyl)-N-(C₁₋₃-alkyl)-amino-carbonyl-methyl, hydroxycarbonylmethyl, C₁₋₃-alkoxy-carbonylmethyl or dimethylaminocarbonylmethyl group,
R₁ is bound in the 4 position of the phenyl group of formula I and denotes
a C₅₋₇-cycloalkylamino group which may be substituted at the amino nitrogen atom by a C₁₋₃-alkyl, C₁₋₃-alkylcarbonyl, amino-C₁₋₃-alkylcarbonyl, carboxy-C₁₋₃-alkylcarbonyl or C₁₋₄-alkoxy-carbonyl-C₁₋₃-alkyl-carbonyl group, a 4- to 7-membered cycloalkyleneiminocarbonyl group
or a 2,5-dihydropyrrol-1-yl-carbonyl group,
R₂ denotes a hydrogen atom or a C₁₋₃-alkyl, ethenyl, ethynyl, trifluoromethyl or trifluoromethoxy group bound in the 3 position or, if R₃ denotes a C₁₋₃-alkyl group, in the 5 position of the phenyl group in formula I or a chlorine or bromine atom bound in the 3 position,
R₃ denotes a hydrogen atom or a C₁₋₃-alkyl group bound in the 2 position of the phenyl group in formula I,
R₄ denotes a hydrogen atom and
Ar denotes a phenyl group disubstituted by the groups R₅ and R₆, while
R₅ is bound in the 3 position if R₆ denotes a hydrogen atom, or is bound in the 5 position if R₆ assumes a meaning other than the hydrogen atom, and denotes an amidino group optionally substituted by a C₁₋₆-alkoxy-carbonyl, 2,2,2-trichloroethoxycarbonyl or phenylcarbonyl group, or a aminomethyl group and
R₆ denotes a hydrogen atom or a hydroxy or C₁₋₃-alkoxy-carbonyloxy group bound in the 2 position,
or a salt thereof.

5. A compound of the formula I according to claim 1, wherein:
(i) m denotes the number 0,
n denotes the number 0 and
A denotes a —CH₂—CH₂— group, or
(ii) m denotes the number 1,
n denotes the number 0 and
A denotes a —CH₂— group,
R₁ denotes a 4- to 7-membered cycloalkyleneiminocarbonyl or 2,5-dihydropyrrol-1-yl-carbonyl group bound in the 4 position of the phenyl group of formula I,
R₂ denotes a hydrogen atom or a substituent selected from fluorine, chlorine, bromine, C₁₋₃-alkyl and trifluoromethyl bound in the 3 position or, if R₃ denotes a C₁₋₃-alkyl group, bound in the 5 position of the phenyl group in formula I,
R₃ denotes a hydrogen atom or a C₁₋₃-alkyl group bound in the 2 position of the phenyl group in formula I,
R₄ denotes a hydrogen atom and
Ar denotes a phenyl group disubstituted by the groups R₅ and R₆, wherein
R₅ is bound in the 5 position and denotes an amidino group optionally substituted by one or two C₁₋₃-alkyl groups, a C₁₋₆-alkoxy-carbonyl or phenylcarbonyl group and
R₆ denotes a hydroxy group bound in the 2 position,
or a salt thereof.

6. A compound selected from the group consisting of:
(1) 2-(5-carbamimidoyl-2-hydroxy-phenyl)-N-[3-methyl-4-(pyrrolidin-1-yl-carbonyl)-phenyl]-ethylamine,
(2) N-(5-carbamimidoyl-2-hydroxy-benzyl)-3-methyl-4-(pyrrolidin-1-yl-carbonyl)-benzylamine,
(3) N-(5-carbamimidoyl-2-hydroxy-benzyl)-2,5-dimethyl-4-(pyrrolidin-1-yl-carbonyl)-benzylamine,
(4) N-(5-carbamimidoyl-2-hydroxy-benzyl)-3-methyl-4-(pyrrolidin-1-yl-carbonyl)-benzamide,
(5) N-(5-carbamimidoyl-2-hydroxy-benzyl)-2,5-dimethyl-4-(pyrrolidin-1-yl-carbonyl)-benzamide,
(6) N-(3-carbamimidoyl-benzyl)-3-methyl-4-(pyrrolidin-1-yl-carbonyl)-benzamide,
(7) N-(5-carbamimidoyl-2-hydroxy-benzyl)-3-trifluoromethyl-4-(pyrrolidin-1-yl-carbonyl)-benzamide,
(8) N-(5-aminomethyl-2-hydroxy-benzyl)-3-methyl-4-(pyrrolidin-1-yl-carbonyl)-benzamide,
(9) 2-(3-aminomethyl-phenyl)-2-[3-methyl-4-(pyrrolidin-1-yl-carbonyl)-phenyl]-acetic acid-N-ethylamide,
(10) 3-(3-aminomethyl-phenyl)-3-[3-methyl-4-(pyrrolidin-1-yl-carbonyl)-benzoylamino]-propionic acid-N-ethylamide,
(11) N-(5-carbamimidoyl-2-hydroxy-benzyl)-3-methyl-4-[N-cyclopentyl-N-(3-ethoxy-carbonyl-propionyl)amino]-benzamide,
(12) N-(5-carbamimidoyl-2-hydroxy-benzyl)-3-methyl-4-(N-acetyl-N-cyclobutyl-amino)-benzamide,
(13) N-(5-carbamimidoyl-2-hydroxy-benzyl)-3-methyl-4-(N-cyclopentyl-N-methyl-amino)-benzamide,
(14) N-(5-carbamimidoyl-2-hydroxy-benzyl)-3-methyl-4-[N-cyclopentyl-N-(3-carboxy-propionyl)-amino]-benzamide,
(15) N-(5-carbamimidoyl-2-hydroxy-benzyl)-4-cyclopentylamino-3-methyl-benzamide, (16) ethyl 2-(3-carbamimidoyl-phenyl)-2-[3-methyl-4-(pyrrolidin-1-yl-carbonyl)-benzoyl-amino]-acetate,
(17) 2-(3-carbamimidoyl-phenyl)-2-[3-methyl-4-(pyrrolidin-1-yl-carbonyl)-benzoyl-amino]-acetic acid,
(18) N-(5-carbamimidoyl-2-hydroxy-benzyl)-3-methyl-4-[N-(2-aminoacetyl)-N-cyclopentyl-amino]-benzamide,
(19) N-(5-carbamimidoyl-2-hydroxy-benzyl)-3-methyl-4-[N-(3-amino-propionyl)-N-cyclopentyl-amino]-benzamide,
(20) N-(5-carbamimidoyl-2-hydroxy-benzyl)-3-chloro-4-(pyrrolidin-1-yI-carbonyl)-benzamide,
(21) ethyl 3-(3-carbamimidoyl-phenyl)-3-[3-methyl-4-(pyrrolidin-1-yl-carbonyl)-benzoyl-amino]-propionate,
(22) ethyl 3-(3-carbamimidoyl-phenyl)-3-[3-chloro-4-(pyrrolidin-1-yl-carbonyl)-benzoyl-amino]-propionate,
(23) ethyl 3-(3-carbamimidoyl-phenyl)-3-{3-methyl-4-[N-(3-amino-propionyl)-N-cyclopentyl-amino]-benzoylamino }-propionate,
(24) ethyl 3-(3-carbamimidoyl-phenyl)-3-[3-bromo-4-(pyrrolidin-1-yl-carbonyl)-benzoyl-amino]-propionate,
(25) ethyl 3-(3-carbamimidoyl-phenyl)-3-[3-methyl-4-(2,5-dihydropyrrol-1-yl-carbonyl)-benzoylamino]-propionate,
(26) ethyl 3-(3-carbamimidoyl-phenyl)-3-[3-ethynyl-4-(pyrrolidin-1-yl-carbonyl)-benzoyl-amino]-propionate,
(27) ethyl 3-(3-carbamimidoyl-phenyl)-3-[3-ethyl-4-(pyrrolidin-1-yl-carbonyl)-benzoyl-amino]-propionate,
(28) ethyl 3-(3-carbamimidoyl-phenyl)-3-[3-ethenyl-4-(pyrrolidin-1-yl-carbonyl)-benzoyl-amino]-propionate,
(29) 3-(3-carbamimidoyl-phenyl)-3-[3-methyl-4-(pyrrolidin-1-yl-carbonyl)-benzoylamino]-propionic acid,
(30) 3-(3-carbamimidoyl-phenyl)-3-[3-trifluoromethyl-4-(pyrrolidin-1-yl-carbonyl)-benzoylamino]-propionic acid,
(31) 3-(3-carbamimidoyl-phenyl)-3-[3-chloro-4-(pyrrolidin-1-yl-carbonyl)-benzoylamino]-propionic acid,
(32) 3-(3-carbamimidoyl-phenyl)-3-[3-methyl-4-(2,5-dihydropyrrol-1-yl-carbonyl)-benzoylamino]-propionic acid,
(33) 3-(3-carbamimidoyl-phenyl)-3-[3-ethynyl-4-(pyrrolidin-1-yl-carbonyl)-benzoyl-amino]-propionic acid,
(34) 3-(3-carbamimidoyl-phenyl)-3-[3-ethyl-4-(pyrrolidin-1-yl-carbonyl)-benzoylamino]-propionic acid,
(35) 3-[3-methyl-4-(pyrrolidin-1-yl-carbonyl)-benzoylamino]-3-(3-carbamimidoyl-phenyl)-propionic acid-N-methyl-N-(hydroxycarbonylmethyl)-amide,
(36) 3-[3-methyl-4-(pyrrolidin-1-yl-carbonyl)-benzoylamino]-3-(3-carbamimidoyl-phenyl)-propionic acid-N-(hydroxycarbonylmethyl)-N-(n-propyl)-amide,
(37) 3-(3-carbamimidoyl-phenyl)-3-[3-ethenyl-4-(pyrrolidin-1-yl-carbonyl)-benzoyl-amino]-propionic acid,
(38) 3-(3-carbamimidoyl-phenyl)-3-[3-methyl-4-(pyrrolidin-1-yl-carbonyl)-benzoyl-amino]-propionic acid-N,N-dimethylamide,
(39) N-[1-(3-carbamimidoyl-phenyl)-2-oxo-2-(pyrrolidin-1-yl)-ethyl]-3-methyl-4-(pyrrolidin-1-yl-carbonyl)-benzamide,
(40) 2-(3-carbamimidoyl-phenyl)-2-[3-methyl-4-(pyrrolidin-1-yl-carbonyl)-benzoyl-amino]-acetic acid-N,N-dimethylamide,
(41) 2-(3-carbamimidoyl-phenyl)-2-[3-methyl-4-(pyrrolidin-1-yl-carbonyl)-benzoyl-amino]-acetic acid-N-ethylamide,
(42) 3-(3-carbamimidoyl-phenyl)-3-[3-methyl-4-(pyrrolidin-1-yl-carbonyl)-benzoyl-amino]-propionic acid-N-ethylamide,
(43) 3-[3-methyl-4-(pyrrolidin-1-yl-carbonyl)-benzoylamino]-3-(3-carbamimidoyl-phenyl)-propionic acid-N-(ethoxycarbonylmethyl)-N-(n-propyl)-amide,
(44) N-[-(5-carbamimidoyl-2-hydroxy-phenyl)-ethyl]-3-methyl-4-(pyrrolidin-1-yl-carbonyl)-benzamide,
(45) N-[1-(5-carbamimidoyl-2-hydroxy-phenyl)-ethyl]-3-bromo-4-(pyrrolidin-1-yl-carbonyl)-benzamide,
(46) N-[1-(5-carbamimidoyl-2-hydroxy-phenyl)-ethyl]-4-(pyrrolidin-1-yl-carbonyl)-benzamide,
(47) ethyl 3-(3-carbamimidoyl-phenyl)-3-[3-trifluoromethyl-4-(pyrrolidin-1-yl-carbonyl)-benzoylamino]-propionate,
(48) N-(5-carbamimidoyl-2-hydroxy-benzyl)-3-trifluoromethoxy-4-(pyrrolidin-1-yl-carbonyl)-benzamide,
(49) 3-(5-carbamimidoyl-2-hydroxy-phenyl)-3-[3-methyl-4-(pyrrolidin-1-yl-carbonyl)-benzoylamino]-propionic acid,
(50) ethyl 3-[3-N-(phenylcarbonyl)-amidino-phenyl]-3-[3-methyl-4-(pyrrolidin-1-yl-carbonyl)-benzoylamino]-propionate,
(51) ethyl 3-[3-N-(n-hexyloxycarbonyl)-amidino-phenyl]-3-[3-methyl-4-(pyrrolidin-1-yl-carbonyl)-benzoylamino]-propionate,
(52) n-propyl 3-[3-N-(phenylcarbonyl)-amidino-phenyl]-3-[3-methyl-4-(pyrrolidin-1-yl-carbonyl)-benzoylamino]-propionate,
(53) ethyl 3-[3-N-(2,2,2-trichloroethyloxycarbonyl)-amidino-phenyl]-3-[3-methyl-4-(pyrrolidin-1-yl-carbonyl)-benzoylamino]-propionate,
(54) N-{5-[N-(n-hexyloxycarbonyl)-amidino]-2-hydroxy-benzyl}-3-methyl-4-(pyrrolidin-1-yl-carbonyl)-benzamide,
(55) N-{5-[N-(phenylcarbonyl)-amidino]-2-hydroxy-benzyl}-3-methyl-4-(pyrrolidin-1-yl-carbonyl)-benzamide,
(56) N-[5-(N-hydroxy-amidino)-2-hydroxy-benzyl]-3-methyl-4-(pyrrolidin-1-yl-carbonyl)-benzamide and
(57) N-{5-[N-(phenylcarbonyl)-amidino]-2-(ethyloxycarbonyloxy)-benzyl}-3-methyl-4-(pyrrolidin-1-yl-carbonyl)-benzamide,
or a derivative thereof wherein at least one amidino group present is substituted by a C₁₋₆-alkoxycarbonyl or phenylcarbonyl group,
or a salt thereof.

7. A physiologically acceptable salt of a compound according to claim 1, 2, 3, 4, 5 or 6, with the exception of those compounds wherein Ar denotes a phenyl or naphthyl group substituted by the groups R₅, R₆ and R₇, and R₅ denotes a cyano group.

8. A pharmaceutical composition a compound according to claim 1, 2, 3, 4, 5 or 6, with the exception of those compounds wherein Ar denotes a phenyl or naphthyl group substituted by the groups R₅, R₆ and R₇, and R₅ denotes a cyano group, or a physiologically acceptable salt thereof, together with one or more inert carriers and/or diluents.

9. A method for treating thrombus formation which method comprises administering to a host in need of such treatment an antithrombotic amount of a compound according to claim 1, 2, 3, 4, 5 or 6, with the exception of those compounds wherein Ar denotes a phenyl or naphthyl group substituted by the groups R₅, R₆ and R₇, and R₅ denotes a cyano group, or a physiologically acceptable salt thereof.
